(19) United States
(12) Patent Application Publication
(10) Pub. No.: US 2026/0218260 A1
(43) Pub. Date: Jul. 30, 2026

(54) OPTIMIZED TAILING OF MESSENGER RNA

(71) Applicant: SANOFI PASTEUR INC., Swiftwater, PA (US)

(72) Inventors: Jonathan ABYSALH, Cambridge, MA (US); Jorel VARGAS, Cambridge, MA (US)

(21) Appl. No.: 19/141,463

(22) PCT Filed: Dec. 22, 2023
(86) PCT No.: PCT/EP2023/087598

§ 371 Date: Jun. 20, 2025

(30) Foreign Application Priority Data
Dec. 23, 2022 (EP) 22315353.7

(51) Int. Cl. C12P 19/34

(52) U.S. Cl. CPC C12P 19/34

(57) ABSTRACT

The present invention relates to a method for tailing in vitro transcribed (IVT) messenger RNA (mRNA) comprising a modified ribonucleotide, said method comprising adding the IVT mRNA to a tailing polymerase in a reaction buffer comprising 30 mM or less of an alkali metal salt and 5 mM or more of a reducing agent.

### RELATED APPLICATIONS

This application is a 35 U.S.C. filing of International Patent Application No. PCT/EP2023/087598, filed Dec. 22, 2023, which claims priority to European Patent Application No. 22315353.7, filed Dec. 23, 2022, the entire disclosures of which are hereby incorporated herein by reference.

### FIELD OF THE INVENTION

The present invention generally relates to methods and compositions for improving the tailing efficiency of in vitro transcribed (IVT) messenger RNA (mRNA) comprising a modified ribonucleotide. In particular, the invention relates to methods and compositions that reduce the percentage of untailed IVT mRNA when a tailing polymerase is used to separately add a tail to the 3′ end after in vitro synthesis of the mRNA.

### BACKGROUND OF THE INVENTION

Messenger RNA (mRNA) as a therapeutic agent is increasingly important. mRNA therapy can restore the normal levels of an endogenous protein or provide an exogenous therapeutic protein without permanently altering the genome sequence or entering the nucleus of the cell. mRNA therapy takes advantage of the cell's own protein production and processing machinery to express a therapeutic peptide, polypeptide, or protein, is flexible to tailored dosing and formulation, and is broadly applicable to any disease or condition that is treatable through the provision of an exogenous protein.

Expression levels of an mRNA-encoded protein can significantly impact the efficacy and therapeutic benefits of mRNA therapy. Effective expression or production of a protein from an mRNA within a cell, and the stability of the mRNA itself, depend on a variety of factors, including the presence of a cap at the 5′ end and an appropriately sized tail at the 3′ end of the mRNA.

The process of manufacturing mRNA for use in therapy typically involves the in vitro transcription of the mRNA from a DNA template. Capping and tailing can occur co-transcriptionally. Alternatively, the in vitro transcribed (IVT) mRNA can be separately capped and/or tailed in subsequent enzymatic reactions.

Methods and compositions have previously been developed to tail IVT mRNA. For example, WO 2021/163134 describes methods for purifying high-quality mRNA suitable for clinical use. The disclosed methods involve capping and tailing mRNA in a reaction buffer having a pH lower than 8.0 and MgCl₂ at a concentration of less than 1.25 mM. WO 2006/029350 describes methods and compositions that enable tagging and amplification of targeted RNA molecules. These methods can involve the enzymatic addition of a polyA tail in a separate reaction.

Achieving high tailing efficiencies, in particular for IVT mRNAs comprising a modified ribonucleotide, remains challenging with existing methods. Therefore, a need exists for methods and compositions that are capable of increasing the tailing efficiency of IVT mRNA.

### SUMMARY OF THE INVENTION

The invention is concerned with a method of tailing in vitro transcribed (IVT) mRNA comprising a modified ribonucleotide. The invention is based on the discovery that adjustment of the tailing conditions can result in a significant improvement of tailing efficiency. In particular, the inventors have discovered that using a reaction buffer comprising 30 mM or less (e.g., 5 mM or less) of an alkali metal salt and 5 mM or more of a reducing agent surprisingly results in higher tailing efficiencies for mRNA comprising a modified ribonucleotide. This is particularly useful when manufacturing mRNA for therapeutic use.

Unless otherwise specified, concentrations of reaction buffer components that are provided herein to describe tailing conditions (e.g., in the context of a tailing method or reaction) refer to working concentrations (i.e., 1× concentrations). A person of skill in the art will appreciate that higher concentrations (typically 10×) are used for storing reaction buffers, and that reaction buffers prepared for storage may therefore require dilution for use in a method or process described herein.

The inventors hypothesized that the presence of a modified ribonucleotide such as N1-methylpseudouridine in mRNA may neutralize the phosphate backbone of the IVT mRNA. This can lead to the formation of complex secondary structures in the IVT mRNA. These secondary structures may interfere with a tailing polymerase accessing the 3′end of the IVT mRNA and initiating tailing. Lowering the concentration of the alkali metal salt present in the reaction buffer reduces its ionic strength. Without wishing to be bound by any particular theory, the lower concentration of the alkali metal salt in the reaction buffer of the invention may reduce the formation of secondary structures in the IVT mRNA, resulting in increased tailing efficiency.

The inventors further hypothesized that secondary structures of the nascent tail may interfere with the activity of the tailing polymerase, resulting in a higher percentage of untailed IVT mRNAs and tailed IVT mRNAs with a longer than desired tail. Without wishing to be bound by any particular theory, adding a reducing agent may reduce the disulfide bonds in the tailing polymerase, potentially resulting in conformational changes that allow the enzyme to handle more complex secondary structures formed by the nascent tail, further increasing the tailing efficiency and resulting in the majority of IVT mRNA having the desired tail length.

In one aspect, the invention relates to a method for tailing in vitro transcribed (IVT) mRNA comprising a modified ribonucleotide, said method comprising adding the IVT mRNA to a tailing polymerase in a reaction buffer comprising 30 mM or less of an alkali metal salt and 5 mM or more of a reducing agent. In some embodiments, the IVT mRNA comprises a 5′ cap. In some embodiments, the 5′ cap was added in a separate reaction, comprising a reaction buffer that was different to the reaction buffer that is used for tailing the IVT mRNA.

In some embodiments, the alkali metal salt in the reaction buffer has a concentration of about 1 mM to about 30 mM. In some embodiments, the alkali metal salt in the reaction buffer has a concentration of about 5 mM or less. In some embodiments, the alkali metal salt in the reaction buffer has a concentration of about 1 mM to about 5 mM.

In some embodiments, the reducing agent in the reaction buffer has a concentration of 5 mM to 50 mM. In some embodiments, the reducing agent in the reaction buffer has a concentration of 5 mM to 20 mM. In specific embodiments, the reducing agent in the reaction buffer has a concentration of about 10 mM.

In some embodiments, the reducing agent is selected from dithiothreitol (DTT), 2-mercaptoethanol (2-ME), and tris(2-carboxyethyl)phosphine (TCEP). In specific embodiments, the reducing agent is dithiothreitol (DTT).

In some embodiments, the alkali metal salt is NaCl or KCl. In specific embodiments, the alkali metal salt is NaCl.

In some embodiments, the modified ribonucleotide is selected from pseudouridine, N1-methylpseudouridine, 5-methylcytidine, and 5-methoxyuridine. In some embodiments, the modified ribonucleotide is a modified uridine. In some embodiments, the modified uridine is N1-methylpseudouridine.

In some embodiments, at least 93% of the IVT mRNA is tailed using a tailing method in accordance with the invention. In some embodiments, at least 94% of the IVT mRNA is tailed. In some embodiments, at least 95% of the IVT mRNA is tailed. In some embodiments, at least 96% of the IVT mRNA is tailed. In some embodiments, at least 97% of the IVT mRNA is tailed. In some embodiments, at least 98% of the IVT mRNA is tailed. In some embodiments, at least 99% of the IVT mRNA is tailed. In some embodiments, 100% of the IVT mRNA is tailed.

In some embodiments, the mRNA tail comprises between about 100 and about 800 ribonucleotides. In some embodiments, the mRNA tail comprises between about 100 and about 500 ribonucleotides. In some embodiments, the mRNA tail comprises between about 100 and about 250 ribonucleotides. In some embodiments, the mRNA tail comprises about 100 or about 200 ribonucleotides.

In some embodiments, the reaction buffer maintains a pH of between about pH 7 and about pH 8. In specific embodiments, the reaction buffer maintains a pH of about pH 7.5.

In some embodiments, the reaction buffer maintains the pH with a buffering reagent. In some embodiments, the buffering reagent is selected from Tris, HEPES, MOPS, acetate, citrate, and phosphate. In some embodiments, the buffering reagent is present at a concentration of between about 5 mM and about 100 mM. In some embodiments, the buffering reagent is present at a concentration of between about 10 mM and about 50 mM. In specific embodiments, the buffering reagent is present at a concentration of about 50 mM.

In some embodiments, the reaction buffer comprises a divalent cation. In some embodiments, the divalent cation is selected from Mg²⁺ and Mn²⁺. In some embodiments, the divalent cation is present at a concentration of between about 5 mM and about 20 mM. In specific embodiments, the divalent cation is at a concentration of between about 5 mM and about 10 mM. In a particular embodiment, the divalent cation is at a concentration of about 10 mM.

In some embodiments, the IVT mRNA does not contain a modified ribonucleotide at the 3′ end. In some embodiments, the IVT mRNA does not contain a modified uridine at the 3′ end. In specific embodiments, the IVT mRNA does not contain a N1-methylpseudouridine at the 3′ end.

In some embodiments, the tailing polymerase is a polyA polymerase. In some embodiments, the polyA polymerase is a bacterial polyA polymerase or a yeast polyA polymerase. In specific embodiments, the polyA polymerase is an E. coli polyA polymerase.

In some embodiments, the reaction buffer includes a suitable concentration of ATP. In some embodiments, ATP is present at a concentration of between about 0.1 mM and about 10 mM.

In a further aspect, the invention relates to compositions comprising tailed IVT mRNA obtainable by a tailing method of the invention. These compositions are characterized by low or undetectable amounts of untailed IVT mRNA and a narrow size distribution of the tailed IVT mRNAs. For example, in some embodiments, compositions comprising tailed IVT mRNA obtainable by a tailing method of the invention comprise less than 5% (e.g., 2% or less) of untailed IVT mRNA as determined by the area under the curve of each species in a capillary gel electropherogram. Moreover, the average tail length is typically close to the desired tail length, e.g., within about 25%, about 20%, about 15%, about 10%, or about 5% of the desired tail length (as determined, e.g., by capillary gel electrophoresis).

In a further aspect, the invention relates to a reaction buffer for use in a method for tailing in vitro transcribed (IVT) mRNA comprising 30 mM or less of an alkali metal salt and 5 mM or more of a reducing agent. The reaction buffer is optimized for tailing IVT mRNA comprising a modified ribonucleotide. The inventors found that this reaction buffer can also be used with IVT mRNA composed solely of unmodified ribonucleotides.

In some embodiments, the alkali metal salt in the reaction buffer has a concentration of about 1 mM to about 30 mM. In some embodiments, the alkali metal salt in the reaction buffer has a concentration of about 5 mM or less. In some embodiments, the alkali metal salt in the reaction buffer has a concentration of about 1 mM to about 5 mM.

In some embodiments, the reducing agent in the reaction buffer has a concentration of 5 mM to 50 mM. In some embodiments, the reducing agent in the reaction buffer has a concentration of 5 mM to 20 mM. In specific embodiments, the reducing agent in the reaction buffer has a concentration of about 10 mM.

In some embodiments, the reducing agent is selected from dithiothreitol (DTT), 2-mercaptoethanol (2-ME), and tris(2-carboxyethyl)phosphine (TCEP). In specific embodiments, the reducing agent is dithiothreitol (DTT).

In some embodiments, the alkali metal salt in the reaction buffer is NaCl or KCl. In specific embodiments, the alkali metal salt is NaCl.

In some embodiments, the reaction buffer has a pH of between about pH 7 and about pH 8. In specific embodiments, the reaction buffer has a pH of about pH 7.5.

In some embodiments, the reaction buffer comprises Tris, HEPES, MOPS, acetate, citrate, or phosphate as a buffering reagent. In some embodiments, the buffering reagent is present at a concentration of between about 5 mM and about 100 mM. In some embodiments, the buffering reagent is present at a concentration of between about 10 mM and about 50 mM. In specific embodiments, the buffering reagent is present at a concentration of about 50 mM.

In some embodiments, the reaction buffer comprises a divalent cation. In some embodiments, the divalent cation is selected from Mg²⁺ and Mn²⁺. In some embodiments, the reaction buffer comprises MgCl₂ or MnCl₂.

In some embodiments, the divalent cation in the reaction buffer is present at a concentration of between about 5 mM and about 20 mM. In specific embodiments, the divalent cation is at a concentration of about 10 mM.

In a further aspect, the invention also relates to a composition comprising in vitro transcribed (IVT) mRNA in a reaction buffer as defined in the preceding paragraphs.

In yet a further aspect, the invention relates to a reaction buffer for use in a method for tailing in vitro transcribed (IVT) mRNA comprising 300 mM or less of an alkali metal salt and 50 mM or more of a reducing agent. This reaction buffer is suitable for shipping and storage and is diluted 10-fold prior to use.

In some embodiments, the alkali metal salt in the reaction buffer has a concentration of about 50 mM or less.

In some embodiments, the reducing agent in the reaction buffer has a concentration of 50 mM to 500 mM. In some embodiments, the reducing agent in the reaction buffer has a concentration of 50 mM to 200 mM. In specific embodiments, the reducing agent in the reaction buffer has a concentration of about 100 mM.

In some embodiments, the reducing agent is selected from dithiothreitol (DTT), 2-mercaptoethanol (2-ME), and tris(2-carboxyethyl)phosphine (TCEP). In specific embodiments, the reducing agent is dithiothreitol (DTT).

In some embodiments, the alkali metal salt is NaCl or KCl. In specific embodiments, the alkali metal salt is NaCl.

In some embodiments, the reaction buffer has a pH of between about pH 7 and about pH 8. In specific embodiments, the reaction buffer has a pH of about pH 7.5.

In some embodiments, the reaction buffer comprises Tris, HEPES, MOPS, acetate, citrate, or phosphate as a buffering reagent. In some embodiments, the buffering reagent is present at a concentration of between about 50 mM and about 1000 mM. In some embodiments, the buffering reagent is present at a concentration of between about 100 mM and about 500 mM. In specific embodiments, the buffering reagent is present at a concentration of about 500 mM.

In some embodiments, the reaction buffer comprises a divalent cation. In some embodiments, the divalent cation is selected from Mg²⁺ and Mn²⁺. In some embodiments, the divalent cation comprises MgCl₂ or MnCl₂.

In some embodiments, the divalent cation is present at a concentration of between about 50 mM and about 200 mM. In specific embodiments, the divalent cation is at a concentration of about 100 mM.

The inventors surprisingly found that tailing IVT mRNA comprising a modified ribonucleotide can be improved by providing untailed IVT mRNA that does not comprise the modified ribonucleotide at the 3′ end. Accordingly, in a further aspect, the invention relates to a method for generating an in vitro transcribed (IVT) mRNA comprising a modified ribonucleotide, the method comprising:
(i) preparing a DNA template wherein the terminal 3′ residue of the DNA template does not encode a modified ribonucleotide of the IVT mRNA;
   and
(ii) transcribing the DNA template with an RNA polymerase in an in vitro transcription (IVT) reaction comprising the modified ribonucleotide.

In some embodiments, the DNA template is a circular vector comprising a restriction site. In some embodiments, step (i) comprises cutting the circular vector at the restriction site to yield the terminal 3′ residue of the DNA template that does not encode a modified ribonucleotide of the IVT mRNA. In some embodiments, the restriction site is cut by BspQI.

In some embodiments, a method for generating IVT mRNA further comprises a step of tailing the IVT mRNA. In some embodiments, the step of tailing comprises adding the IVT mRNA to a tailing polymerase in a reaction buffer comprising 30 mM or less of an alkali metal salt (e.g., NaCl) and 5 mM or more of a reducing agent (e.g., DTT). In some embodiments, the alkali metal salt in the reaction buffer has a concentration of 5 mM or less.

In a further aspect, the invention relates to a method for tailing in vitro transcribed (IVT) mRNA comprising a modified ribonucleotide, comprising:
(i) providing untailed IVT mRNA which does not comprise the modified ribonucleotide at the 3′ end; and
(ii) adding a reaction buffer and a tailing polymerase.

In some embodiments, the reaction buffer comprises 30 mM or less of an alkali metal salt (e.g., NaCl) and 5 mM or more of a reducing agent (e.g., DTT). In specific embodiments, the alkali metal salt in the reaction buffer has a concentration of about 5 mM or less.

In some embodiments, the reducing agent in the reaction buffer has a concentration of 5 mM to 50 mM. In some embodiments, the reducing agent in the reaction buffer has a concentration of 5 mM to 20 mM. In specific embodiments, the reducing agent in the reaction buffer has a concentration of about 10 mM.

In some embodiments, the reaction buffer comprises a divalent cation (e.g., Mg²). In some embodiments, the divalent cation is present at a concentration of between about 5 mM and about 20 mM. In specific embodiments, the divalent cation is at a concentration of about 10 mM.

Other features, objects, and advantages of the present invention are apparent in the detailed description, drawings and embodiments that follow. It should be understood, however, that the detailed description, the drawings, and the embodiments, while indicating embodiments of the present invention, are given by way of illustration only, not limitation. Various changes and modifications will be apparent to those skilled in the art.

### BRIEF DESCRIPTION OF THE DRAWINGS

Embodiments of the invention will be described, by way of example, with reference to the following drawings.

FIGS. 1A-3C illustrate that a reaction buffer comprising 30 mM or less of an alkali metal salt and 5 mM or more of a reducing agent improves tailing efficiency and significantly reduces the percentage of untailed IVT mRNAs comprising a modified ribonucleotide. FIGS. 1A-3C show separation of IVT mRNA A (FIGS. 1A-1C), mRNA B (FIGS. 2A-2C) and mRNA C (FIGS. 3A-3C) by capillary electrophoresis. The x-axis indicates the length of each IVT mRNA as the number of ribonucleotides. The y-axis shows relative fluorescent units (RFU). In FIGS. 1A, 2A, and 3A, the IVT mRNAs were tailed in a reaction buffer comprising 250 mM of an alkali metal salt and no reducing agent (control buffer in Table 2). In FIGS. 1B, 2B, and 3B, the IVT mRNAs were tailed in a reaction buffer comprising 5 mM of the alkali metal salt and no reducing agent (buffer 8 in Table 2). In FIGS. 1C, 2C, and 3C, the IVT mRNAs were tailed in a reaction buffer comprising 5 mM of the alkali metal salt and 10 mM of a reducing agent (buffer 8 in Table 2 with 10 mM DTT). Use of the control buffer resulted in a clearly identifiable additional peak preceding the main peak (marked by an arrow), indicating a significant amount of untailed IVT mRNA (see FIGS. 1A, 2A, and 3A). Reduction of the alkali metal salt concentration markedly reduced the size of the additional peak preceding the main peak (see the arrow in FIGS. 1B, 2B, and 3B), as well as the average tail length, as the main peak is shifted to the left in FIGS. 1B, 2B, and 3B as compared to the main peak in FIGS. 1A, 2A, and 3A. When a reducing agent was further added to the buffer comprising a reduced alkali metal salt concentration, the additional peak was no longer discernible and the average tail length was further decreased (see FIGS. 1C, 2C, and 3C). Indeed, the main peak is shifted to the left in FIGS. 1C, 2C, and 3C as compared to the main peak in FIGS. 1A-1B, 2A-2B, and 3A-3B.

FIG. 4 illustrates that an optimized reaction buffer comprising 5 mM or less of an alkali metal salt and 5 mM or more of a reducing agent improves tailing efficiency, independent of the ribonucleotide at the 3′ end of the IVT mRNA or its ribonucleotide sequence. Seven different IVT mRNAs were prepared either from a DNA template plasmid with backbone (I) cut with HindIII (labelled as ‘HindIII-cut template’) or a DNA template plasmid with backbone (II) cut with BspQI (labelled as ‘BspQI-cut template’). Tailing of each IVT mRNA was performed in a reaction buffer comprising 250 mM of an alkali metal salt and no reducing agent (labelled as buffer ‘C’, corresponding to the control buffer provided in Table 2) or in a reaction buffer comprising 5 mM of the alkali metal salt and 10 mM of a reducing agent (labelled as buffer ‘O’, corresponding to buffer 8 provided in Table 2 with DTT). The desired tail length is 500 nucleotides for IVT mRNAs 1-6 and 200 nucleotides for IVT mRNA 7. IVT-mRNAs produced with the Hind-III-cut template had a modified ribonucleotide (N1-methylpseudouridine) at the 3′ end. The bar graphs show the percentage of untailed IVT mRNA for each construct after tailing in the control buffer (‘C’) or in buffer 8 with DTT (‘O’). For each IVT mRNA, performing the tailing reaction in buffer 8 with DTT improved the tailing efficiency compared to performing the tailing reaction in the control buffer. mRNA that was in vitro transcribed from a BspQI-cut template resulted in a lower percentage of untailed mRNAs compared to mRNA transcribed from a HindIII-cut template.

FIG. 5 illustrates that an optimized reaction buffer of the invention can be used for tailing IVT mRNA comprising a modified ribonucleotide at a large scale (>1 g of IVT mRNA per batch), with the resulting average tail length being close to the desired length. Using capillary gel electrophoresis, ten 10 g batches of tailed IVT mRNA prepared with the unoptimized control buffer described in Table 2 (buffer ‘C’) were compared to four 16 g batches prepared with buffer 8 with 10 mM DTT (buffer ‘O’). The desired tail length was 200 nucleotides. The average tail length achieved with buffer ‘C’ was 45-50% longer than the desired tail length for most of the tested batches (9 out of 10). In contrast, using buffer ‘O’, the average tail length was only about 5% longer than the desired tail length for three of the four tested batches. Even for the worst-performing batch, the average tail length was only about 25% longer than the desired tail length, when buffer ‘O’ was used. The nucleic acid sequence of the IVT mRNA did not affect tailing efficiency with buffer ‘O’.

### DEFINITIONS

In order for the present invention to be more readily understood, certain terms are first defined below. Additional definitions for the following terms and other terms are set forth throughout the specification.

As used in this specification and the appended claims, the singular forms “a,” “an” and “the” include plural referents unless the context clearly dictates otherwise. For example, “a ribonucleotide” is understood to represent one or more ribonucleotides. As such, the terms “a” (or “an”), “one or more”, and “at least one” can be used interchangeably herein.

Unless specifically stated or obvious from context, as used herein, the term “or” is understood to be inclusive and covers both “or” and “and”. Furthermore, “and/or” where used herein is to be taken as specific disclosure of each of the two specified features or components with or without the other. Thus, the term “and/or” as used in a phrase such as “A and/or B” herein is intended to include “A and B”, “A or B”, “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising”, otherwise analogous aspects described in terms of “consisting of” and/or “consisting essentially of” are also provided.

As used herein, the term “about” refers to an interval of accuracy that a person skilled in the art will understand to still ensure the technical effect of the feature in question. The term indicates a deviation from the indicated numerical value of ±10%. In some embodiments, the deviation is ±5% of the indicated numerical value. In certain embodiments, the deviation is 10% of the indicated numerical value.

As used herein, the term “mRNA” refers to a polyribonucleotide that encodes at least one polypeptide. mRNA as used herein encompasses both modified and unmodified RNA. mRNA may contain one or more coding and non-coding regions (e.g., a 5′ untranslated region and a 3′ untranslated region). mRNA can be purified from natural sources, produced using recombinant expression systems and optionally purified, in vitro transcribed, or chemically synthesized. The invention particularly relates to in vitro transcribed (IVT) mRNA. Where appropriate, e.g., in the case of chemically synthesized molecules, mRNA can comprise nucleoside analogues such as analogues having chemically modified bases or sugars, backbone modifications, etc. An mRNA sequence is presented in the 5′ to 3′ direction unless otherwise indicated. A typical mRNA comprises a 5′ cap, a 5′ untranslated region (5′ UTR), a protein-coding region, a 3′ untranslated region (3′ UTR), and a 3′ tail. In some embodiments, the tail structure is a poly(C) tail. More typically, the tail structure is a polyA tail.

As used herein the term “sequence-optimized” is used to describe a nucleotide sequence that is modified relative to a naturally-occurring or wild-type nucleotide sequence. Such modifications may include, e.g., codon optimization and/or the use of 5′ UTRs and 3′ UTRs which are not normally associated with the naturally-occurring or wild-type nucleic acid. As used herein, the terms “codon optimization” and “codon-optimized” refer to modifications of the codon composition of a naturally-occurring or wild-type nucleic acid encoding a peptide, polypeptide or protein that do not alter its amino acid sequence, thereby improving protein expression of said nucleic acid. In the context of the present invention, “codon optimization” may also refer to the process by which one or more optimized nucleotide sequences are arrived at by removing, with filters, less than optimal nucleotide sequences from a list of nucleotide sequences, such as filtering by guanine-cytosine content, codon adaptation index, presence of destabilizing nucleic acid sequences or motifs, and/or presence of pause sites and/or terminator signals.

As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

As used herein, the term “template DNA” (or “DNA template”) relates to a DNA molecule comprising a nucleic acid sequence encoding an mRNA transcript to be synthesized by in vitro transcription. The template DNA is used as template for in vitro transcription in order to produce the mRNA transcript encoded by the template DNA. The template DNA comprises all elements necessary for in vitro transcription, particularly a promoter element for binding of a DNA-dependent RNA polymerase, such as, e.g., T3, T7 or SP6 RNA polymerase, which is operably linked to the DNA sequence encoding a desired mRNA transcript. Furthermore, the template DNA may comprise primer binding sites 5′ and/or 3′ of the DNA sequence encoding the mRNA transcript to determine the identity of the DNA sequence encoding the mRNA transcript, e.g., by PCR or DNA sequencing. The “template DNA” in the context of the present invention may be a linear or a circular DNA molecule. As used herein, the term “template DNA” may refer to a DNA vector, such as a plasmid DNA, which comprises a nucleic acid sequence encoding the desired mRNA transcript.

As used herein, the term “tailing efficiency” refers to how effective the tailing polymerase is at adding a tail to IVT mRNA. Typically, an efficient tailing reaction results in the IVT mRNA having an average tail length that is close to the desired tail length. The desired tail length correlates to the expected average tail length under optimal tailing conditions. Therefore, an increase in tailing efficiency usually results in both a lower percentage of untailed IVT mRNA and an average tail length close to the indicated desired tail length.

Unless otherwise defined herein, technical and scientific terms used herein have the same meaning as commonly understood by one of ordinary skill in the art to which this application belongs and as commonly used in the art to which this application belongs. Exemplary methods and materials are described below, although methods and materials similar or equivalent to those described herein can also be used in the practice or testing of the present disclosure. In case of conflict, the present specification, including definitions, will control.

Generally, nomenclature used in connection with, and techniques of, cell and tissue culture, molecular biology, virology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular.

Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers.

All publications and other reference materials referenced herein are hereby incorporated by reference in their entirety. Although a number of documents are cited herein, this citation does not constitute an admission that any of these documents forms part of the common general knowledge in the art.

### DETAILED DESCRIPTION OF THE INVENTION

The present invention relates to a method for tailing in vitro transcribed (IVT) messenger RNA (mRNA) comprising a modified ribonucleotide. The invention is based on the discovery that adjustment of the tailing conditions can result in a significant improvement of tailing efficiency. In particular, using a reaction buffer comprising 5 mM or less of an alkali metal salt and 5 mM or more of a reducing agent was found to result in higher tailing efficiencies, even when the IVT mRNA had a terminal modified ribonucleotide at the 3′ end.

The inventors additionally found that tailing efficiency can be improved if the DNA template has a terminal 3′ residue that does not encode a modified ribonucleotide of the IVT mRNA. Accordingly, in some aspects, the invention also relates to a method for tailing in vitro transcribed (IVT) messenger RNA (mRNA) comprising a modified ribonucleotide, comprising:
(i) providing untailed IVT mRNA which does not comprise the modified ribonucleotide at the 3′ end; and
(ii) adding a reaction buffer and a tailing polymerase.
   IVT mRNA

### In Vitro Transcription

Various methods for synthesizing mRNA via in vitro transcription (IVT) are described in US Patent Publication No. US 2018/0258423 and International Patent Publication No. WO 2021/168052A1, which are incorporated herein by reference, and can be used to practice the present invention. Briefly, IVT is typically performed with a reaction mixture comprising DNA template containing a promoter, a pool of ribonucleotide triphosphates, a buffer system (that may include DTT and magnesium ions), and an appropriate RNA polymerase (e.g., T3, T7, or SP6 RNA polymerase). The DNA template is typically linearized with a suitable restriction enzyme prior to the IVT reaction. The IVT reaction can be terminated by the addition of DNase I, which digests the DNA template. The exact conditions will vary according to the specific application.

### DNA Template

A typical DNA template in accordance with the invention comprises a promoter sequence, for example a T3, T7, or SP6 promoter, followed by the nucleotide sequence for the desired mRNA. The nucleotide sequence usually comprises a 5′ untranslated region (5′ UTR), a coding region for a polypeptide of interest, and a 3′ untranslated region (3′ UTR).

In some embodiments, the nucleotide sequence comprises a 5′ untranslated region (5′ UTR) different from the 5′ UTR present in a naturally occurring mRNA encoding the polypeptide of interest.

In some embodiments, the nucleotide sequence comprises a 3′ untranslated region (3′ UTR) different from the 3′ UTR present in a naturally occurring mRNA encoding the polypeptide of interest.

For example, suitable 5′ and 3′ UTRs are described in WO2012/075040, which is incorporated herein by reference.

In certain embodiments, the 5′ and/or 3′ UTR sequences can be derived from mRNA which are stable (e.g., globin, actin, GAPDH, tubulin, histone, or citric acid cycle enzymes) to increase the stability of the mRNA. For example, a 5′ UTR sequence may include a partial sequence of a CMV immediate-early 1 (IE1) gene, or a fragment thereof, to improve the nuclease resistance and/or improve the half-life of the mRNA. Also contemplated is the inclusion of a sequence encoding human growth hormone (hGH), or a fragment thereof, to the 3′ end or untranslated region of the mRNA. Exemplary 5′ UTRs include a sequence derived from a CMV immediate-early 1 (IE1) gene (U.S. Publication Nos. 2014/0206753 and 2015/0157565, each of which is incorporated herein by reference), or the sequences provided in Example 1 of U.S. Publication No. 2016/0151409, incorporated herein by reference.

In various embodiments, the 5′ UTR may be derived from the 5′ UTR of a TOP gene. TOP genes are typically characterized by the presence of a 5′-terminal oligopyrimidine (TOP) tract. Furthermore, most TOP genes are characterized by growth-associated translational regulation. However, TOP genes with a tissue specific translational regulation are also known. In certain embodiments, the 5′ UTR derived from the 5′ UTR of a TOP gene lacks the 5′ TOP motif (the oligopyrimidine tract) (e.g., U.S. Publication Nos. 2017/0029847, 2016/0304883, 2016/0235864, and 2016/0166710, each of which is incorporated herein by reference).

In certain embodiments, the 5′ UTR is derived from a ribosomal protein Large 32 (L32) gene (U.S. Publication No. 2017/0029847, supra).

In certain embodiments, the 5′ UTR is derived from the 5′ UTR of a hydroxysteroid (17-b) dehydrogenase 4 gene (HSD17B4) (U.S. Publication No. 2016/0166710, supra).

In certain embodiments, the 5′ UTR is derived from the 5′ UTR of an ATP5A1 gene (U.S. Publication No. 2016/0166710, supra).

In some embodiments, an internal ribosome entry site (IRES) is used instead of a 5′ UTR.

The IVT mRNA is typically transcribed from a DNA template which is linearized using a restriction enzyme. In this context, any restriction enzyme (see e.g., Roberts et al. (2015) Nucl. Acids Res. 43;D1:D298-D299) may be used. Generally, the restriction enzyme is a type II restriction enzyme, such as a type IIP or type IIS restriction enzyme. In some embodiments, the restriction enzyme is EcoRI, BciVI, Spel, Xbal, Ndel, Aflll, Sacl, Kpnl, Smal, BamHI, Sail, Sbfl, Pstl, BspQI, or Hindlll.

In some embodiments, the restriction site for linearizing the DNA template is selected so that the resulting IVT mRNA does not include a modified ribonucleotide as the terminal nucleotide at the 3′ end. Accordingly, in some aspects, the invention also relates to a method for generating an in vitro transcribed (IVT) messenger RNA (mRNA) comprising a modified ribonucleotide, wherein the method comprises:
(i) preparing a DNA template wherein the terminal 3′ residue of the DNA template does not encode a modified ribonucleotide of the IVT mRNA;
   and
(ii) transcribing the DNA template with an RNA polymerase in an in vitro transcription reaction comprising the modified ribonucleotide.

In some embodiments, the DNA template is a circular vector comprising a restriction site. In some embodiments, step (i) comprises cutting the circular vector at the restriction site to yield the terminal 3′ residue of the DNA template that does not encode a modified ribonucleotide of the IVT mRNA. In some embodiments, the restriction site is cut by BspQI. Accordingly, in some embodiments, a suitable restriction enzyme for preparing the DNA template in step (i) is BspQI. In some embodiments, the IVT mRNA does not comprise N1-methylpseudouridine as the terminal 3′ residue.

### Ribonucleotides

In accordance with the invention, the IVT mRNA is a modified RNA, wherein the modification refers to chemical or biological modifications comprising backbone modifications, sugar modifications, or base modifications. A backbone modification is a modification in which phosphates of the backbone of the nucleotides of the RNA are chemically modified (e.g., phosphorothioates and 5′-N-phosphoramidite linkages). A sugar modification is a chemical modification of the sugar of the nucleotides of the RNA (e.g., 2′-fluororibose, ribose, 2′-deoxyribose, arabinose, and hexose). A base modification is a chemical modification of the base moiety of the nucleotides of the RNA.

In a particular embodiment, the IVT mRNA comprises a modified ribonucleotide, such as ribonucleotide analogue (e.g., adenosine analogue, guanosine analogue, cytidine analogue, and/or uridine analogue). The presence of a modified ribonucleotide may render the mRNA more stable and/or less immunogenic than a control mRNA with the same sequence but containing only naturally-occurring ribonucleotides.

The modified ribonucleotide typically takes the place of a naturally occurring nucleotide. Accordingly, the IVT mRNA of the invention comprises both unmodified and modified ribonucleotides. Such IVT mRNA can be prepared by including a modified ribonucleoside in the IVT reaction mixture, typically in place of a naturally occurring ribonucleoside (e.g., N1-methylpseudouridine in place of uridine). This results in IVT mRNA in which 100% of the naturally occurring ribonucleotide is replaced by a corresponding modified ribonucleotide (e.g., 100% of the uridines are replaced with N1-methyl-pseudouridine). In some embodiments, only a portion of the naturally occurring ribonucleoside (e.g., at least 1%, 5%, 10%, 15%, 20% or 25% of the naturally occurring ribonucleoside) is replaced with a modified ribonucleoside. In some embodiments, one or more naturally occurring ribonucleosides is replaced with a modified ribonucleoside. For example, two or more ribonucleosides may be modified ribonucleosides (e.g., uridines may be replaced with 2-thio-uridine and cytidines may be replaced with 5-methylcytidine). For example, 25% of the uridines may be replaced with 2-thio-uridine and/or 25% of cytidine residues may be replaced with 5-methylcytidine.

In some embodiments, the modified ribonucleoside comprises at least one modification selected from a modified sugar and a modified nucleobase relative to the corresponding naturally occurring ribonucleoside.

The modified ribonucleoside can be a modified uridine, cytidine, adenosine, or guanosine. Some exemplary chemical modifications of ribonucleosides in the mRNA molecule include, e.g., pyridine-4-one ribonucleoside, 5-aza-uridine, 2-thio-5-aza-uridine, 2-thiouridine, 4-thio pseudouridine, 2-thio-pseudouridine, 5-hydroxyuridine, 3-methyluridine, 5-carboxymethyl uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N⁶-methyladenosine, N⁶-isopentenyladenosine, N⁶-(cis-hydroxyisopentenyl) adenosine, 2-methylthio-N⁶-(cis-hydroxyisopentenyl) adenosine, N⁶-glycinylcarbamoyladenosine, N⁶-threonylcarbamoyladenosine, 2-methylthio-N⁶-threonyl carbamoyladenosine, N⁶,N⁶-dimethyladenosine, 7-methyladenine, 2-methylthioadenine, 2-methoxyadenine, inosine, 1-methyl-inosine, wyosine, wybutosine, 7-deaza guanosine, 7-deaza-8-aza-guanosine, 6-thio guanosine, 6-thio-7-deazaguanosine, 6-thio-7-deaza-8-aza-guanosine, 7-methyl-guanosine, 6-thio-7-methylguanosine, 7-methylinosine, 6-methoxy guanosine, 1-methylguanosine, N²-methylguanosine, N²,N²-dimethylguanosine, 8-oxo-guanosine, 7-methyl-8-oxo-guanosine, 1-methyl-6-thio-guanosine, N²-methyl-6-thio-guanosine, and N²,N²-dimethyl-6-thio-guanosine.

In some embodiments, the modified ribonucleoside is a modified uridine selected from pseudouridine, pyridine-4-one ribonucleoside, 5-aza-uridine, 6-aza-uridine, 2-thio-5-aza-uridine, 2-thio-uridine, 4-thio-uridine, 4-thio-pseudouridine, 2-thio-pseudouridine, 5-hydroxy uridine, 5-aminoallyl-uridine, 5-halo-uridine (e.g., 5-iodom uridine or 5-bromo uridine), 3-methyl uridine, 5-methoxy-uridine, uridine-5-oxyacetic acid, uridine-5-oxyacetic acid methyl ester, 5-carboxymethyl-uridine, 1-carboxymethyl-pseudouridine, 5-carboxyhydroxymethyl uridine, 5-carboxyhydroxymethyl-uridine methyl ester, 5-methoxycarbonylmethyluridine, 5-methoxycarbonylmethyl-2-thio-uridine, 5-aminomethyl-2-thiouridine, 5-methylaminomethyl uridine, 5-methylaminomethyl-2-thio-uridine, 5-methylaminomethyl-2-selenouridine, 5-carbamoylmethyl-uridine, 5-carboxymethylaminomethyl-uridine, 5-carboxymethylaminomethyl-2-thio-uridine, 5-propynyl-uridine, 1-propynyl-pseudouridine, 5-taurinomethyl-uridine, 1-taurinomethyl pseudouridine, 5-taurinomethyl-2-thio-uridine, 1-taurinomethyl-4-thio-pseudouridine, 5-methyl-uridine (m⁵U, e.g., having the nucleobase deoxythymine), 1-methyl-pseudouridine, 5-methyl-2-thio-uridine, 1-methyl-4-thio-pseudouridine, 4-thio-1-methyl-pseudouridine, 3-methyl-pseudouridine), 2-thio-1-methyl-pseudouridine, 1-methyl-1-deaza-pseudouridine, 2-thio-1-methyl-1-deaza-pseudouridine, dihydrouridine, dihydropseudouridine, 5,6-dihydrouridine, 5-methyl-dihydrouridine, 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl) uridine, 1-methyl-3-(3-amino-3-carboxypropyl) pseudouridine, 5-(isopentenylaminomethyl) uridine, 5-(isopentenylaminomethyl)-2-thio-uridine, alpha-thio-uridine, 2′-O-methyl uridine, 5,2′-O-dimethyl uridine, 2′-O-methyl-pseudouridine, 2-thio-2′-O-methyl uridine, 5-methoxycarbonylmethyl-2′-O-methyl uridine, 5-carbamoylmethyl-2′-O-methyl uridine, 5-carboxymethylaminomethyl-2′-O-methyl uridine, 3,2′-O-dimethyl uridine, 5-(isopentenylaminomethyl)-2′-O-methyl uridine, 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino) uridine.

In some embodiments, the modified uridine is selected from N1-methylpseudouridine, pseudouridine, 2-thiouridine, 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine. In some embodiments, the modified uridine is N1-methylpseudouridine.

In some embodiments, the modified ribonucleoside is a modified cytidine selected from 5-aza-cytidine, 6-aza-cytidine, pseudoisocytidine, 3-methylcytidine, N⁴-acetyl cytidine, 5-formyl-cytidine, N⁴-methylcytidine, 5-methylcytidine, 5-halo cytidine (e.g., 5-iodo cytidine), 5-hydroxy methylcytidine, 1-methyl-pseudoisocytidine, pyrrolo-cytidine, pyrrolo-pseudoisocytidine, 2-thio-cytidine, 2-thio-5-methylcytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio zebularine, 2-thio-zebularine, 2-methoxy cytidine, 2-methoxy-5-methylcytidine, 4-methoxy pseudoisocytidine, 4-methoxy-1-methyl-pseudoisocytidine, lysidine, alpha-thio-cytidine, 2′-O-methylcytidine, 5,2′-O-dimethyl cytidine, N⁴-acetyl-2′-O-methylcytidine, N⁴,2′-O-dimethyl cytidine, 5-formyl-2′-O-methylcytidine, N⁴,N⁴,2′-O-trimethyl cytidine, 1-thio-cytidine, 2′-F-ara-cytidine, 2′-F cytidine, and 2′-OH-ara-cytidine.

In some embodiments, the modified ribonucleoside is a modified pyrimidine ribonucleoside. In some embodiments, the modified ribonucleoside is selected from pseudouridine, N1-methylpseudouridine, 5-methylcytidine, 5-methoxyuridine, and any combination thereof. In some embodiments, both cytidine and uracil are replaced with modified nucleosides (e.g., N1-methylpseudouridine and 5-methylcytidine).

In some embodiments, the modified ribonucleoside is a modified purine ribonucleoside. In some embodiments, the modified ribonucleoside is a modified adenosine selected from 2-amino purine, 2,6-diamino purine, 2-amino-6-halo purine (e.g., 2-amino-6-chloro purine), 6-halo purine (e.g., 6-chloro purine), 2-amino-6-methyl purine, 8-azido adenosine, 7-deaza-adenine, 7-deaza-8-aza adenine, 7-deaza-2-amino purine, 7-deaza-8-aza-2-amino purine, 7-deaza-2,6-diamino purine, 7-deaza-8-aza-2,6-diamino purine, 1-methyladenosine, 2-methyl adenine, N⁶-methyladenosine, 2-methylthio-N⁶-methyladenosine, N⁶-isopentenyl adenosine, 2-methylthio-N⁶-isopentenyl adenosine, N⁶-(cis-hydroxyisopentenyl) adenosine, 2-methylthio-N⁶-(cis-hydroxyisopentenyl)adenosine, N⁶-glycinylcarbamoyl adenosine, N⁶-threonylcarbamoyl adenosine, N⁶-methyl-N⁶-threonylcarbamoyl adenosine, 2-methylthio-N⁶-threonylcarbamoyl adenosine, N⁶,N⁶-dimethyl adenosine, N⁶-hydroxynorvalylcarbamoyl adenosine, 2-methylthio-N⁶-hydroxynorvalylcarbamoyl adenosine, N⁶-acetyl adenosine, 7-methyladenine, 2-methylthio-adenine, 2-methoxyadenine, alpha-thio-adenosine, 2′-O-methyladenosine, N⁶,2′-O-dimethyl adenosine, N⁶,N⁶,2′-O-trimethyl adenosine, 1,2′-O-dimethyl adenosine, 2′-O-ribosyl adenosine (phosphate), 2-amino-N⁶-methyl purine, 1-thio-adenosine, 8-azido-adenosine, 2′-F-ara-adenosine, 2′-F adenosine, 2′-OH-ara-adenosine, and N⁶-(19-amino-pentaoxanonadecyl) adenosine.

In some embodiments, the modified ribonucleoside is a modified guanosine selected from inosine, 1-methyl inosine, wyosine, methylwyosine, 4-demethyl wyosine, isowyosine, wybutosine, peroxywybutosine, hydroxywybutosine, undermodified hydroxywybutosine, 7-deaza-guanosine, queuosine, epoxyqueuosine, galactosyl queuosine, mannosyl queuosine, 7-cyano-7-deaza-guanosine, 7-aminomethyl-7-deaza-guanosine, archaeosine, 7-deaza-8-aza-guanosine, 6-thio-guanosine, 6-thio-7-deaza-guanosine, 6-thio-7-deaza-8-aza-guanosine, 7-methylguanosine, 6-thio-7-methylguanosine, 7-methyl inosine, 6-methoxy guanosine, 1-methylguanosine, N²-methyl-guanosine, N²,N²-dimethyl guanosine, N^{2,7}-dimethyl guanosine, N²,N^{2,7}-dimethyl guanosine, 8-oxo-guanosine, 7-methyl-8-oxo-guanosine, 1-methylguanosine, N²-methyl-6-thio-guanosine, N²,N²-dimethyl-6-thio-guanosine, alpha-thio-guanosine, 2′-O-methylguanosine, N²-methyl-2′-O-methylguanosine, N²,N²-dimethyl-2′-O-methylguanosine, 1-methyl-2′-O-methylguanosine, N^{2,7}-dimethyl-2′-O-methylguanosine, 2′-O-methyl inosine, 1,2′-O-dimethyl inosine, 2′-O-ribosyl guanosine (phosphate), 1-thio-guanosine, O⁶-methylguanosine, 2′-F-ara guanosine, and 2′-F guanosine.

In some embodiments, the modified ribonucleoside is a ribonucleoside analogue selected from 2-aminoadenosine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, pseudouridine (e.g., N1-methylpseudouridine), 2-thiouridine, and 2-thiocytidine. See, e.g., U.S. Pat. No. 8,278,036 or WO 2011/012316 for a discussion of 5-methylcytidine, pseudouridine, and 2-thio-uridine and their incorporation into mRNA.

In some embodiments, the modified ribonucleoside is selected from pseudouridine, N1-methylpseudouridine, 2-thiouridine, 4′-thiouridine, 5-methylcytidine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine, and 2′-O-methyl uridine.

In some embodiments, the IVT mRNA may be RNA wherein 25% of uridine residues are 2-thio-uridine and 25% of cytidine residues are 5-methylcytidine. Teachings for the use of such modified RNA are disclosed in US Patent Publication US 2012/0195936 and international publication WO 2011/012316, both of which are hereby incorporated by reference in their entirety. In some embodiments, the IVT mRNA may be RNA where 100% of uridine residues are N1-methylpseudouridine (also occasionally referred to as 1-methylpseudouridine).

### Tailing Conditions

### Alkali Metal Salt

In accordance with the invention, the alkali metal salt in the reaction buffer has a concentration of about 30 mM or less, e.g., about 1 mM to about 30 mM, about 5 mM to about 30 mM, or about 5 mM to about 27.5 mM. In some embodiments, the alkali metal salt in the reaction buffer has a concentration of about 25 mM or less e.g., about 1 mM to about 25 mM, or about 5 mM to about 25 mM. In some embodiments, the alkali metal salt in the reaction buffer has a concentration of about 20 mM or less, e.g., about 1 mM to about 20 mM or about 5 mM to about 20 mM. In some embodiments, the alkali metal salt in the reaction buffer has a concentration of about 15 mM or less, e.g., about 1 mM to about 15 mM or about 5 mM to about 15 mM. In some embodiments, the alkali metal salt in the reaction buffer has a concentration of about 10 mM or less, e.g., about 1 mM to about 10 mM or about 5 mM to about 10 mM.

In some embodiments, the alkali metal salt in the reaction buffer has a concentration of about 5 mM or less, e.g., about 1 mM to about 5 mM. In specific embodiments, the alkali metal salt has a concentration of about 5 mM. In other embodiments, the alkali metal salt has a concentration of about 4 mM, about 3 mM, about 2 mM, or about 1 mM.

In some embodiments, the alkali metal salt in the reaction buffer is NaCl. In some embodiments, the alkali metal salt in the reaction buffer is KCl.

In some embodiments, the alkali metal salt in the reaction buffer is NaCl and has a concentration of about 5 mM or less, e.g., about 1 mM to about 5 mM, such as about 4 mM, about 3 mM, or about 2 mM. In a specific embodiment, the alkali metal salt in the reaction buffer is NaCl and has a concentration of about 5 mM.

In some embodiments, the alkali metal salt in the reaction buffer is KCl and has a concentration of about 5 mM or less, e.g., about 1 mM to about 5 mM, such as about 4 mM, about 3 mM, or about 2 mM. In a specific embodiment, the alkali metal salt in the reaction buffer is KCl and has a concentration of about 5 mM.

### Reducing Agent

The inventors found that the addition of a reducing agent can further increase tailing efficiency when the reaction buffer comprises 30 mM or less (e.g., 5 mM or less) of an alkali metal salt. A concentration of at least 5 mM of a reducing agent was found to be effective for this purpose. Thus, in some embodiments, the reaction buffer comprises a reducing agent.

In some embodiments, the reducing agent has a concentration of about 5 mM to about 50 mM. In some embodiments, the reducing agent has a concentration of about 5 mM to about 20 mM. In some embodiments, the reducing agent has a concentration of about 5 mM, about 10 mM, about 15 mM, or about 20 mM. In specific embodiments, the reducing agent has a concentration of about 10 mM.

In some embodiments, the reducing agent reduces disulphide bonds. Suitable reducing agent include dithiothreitol (DTT), 2-mercaptoethanol (2-ME), and tris(2-carboxyethyl)phosphine (TCEP).

In a specific embodiment, the reducing agent is DTT. In some embodiments, the reaction buffer comprises DTT at a concentration of about 5 mM to about 50 mM. In a specific embodiment, the reaction buffer comprises DTT at a concentration of about 10 mM.

pH

In some embodiments, the reaction buffer maintains a pH of between about 6 and about 8.5. In some embodiments, the reaction buffer maintains a pH of between about 7 and pH 8 (e.g., between about 7.2 and 7.8). In some embodiments, the reaction buffer maintains a pH of about 7.0, about 7.1, about 7.2, about 7.3, about 7.4, about 7.5, about 7.6, about 7.7, about 7.8, about 7.9, or about 8.0. In a specific embodiment, the reaction buffer maintains a pH of about 7.5.

In some embodiments, the reaction buffer maintains the pH (e.g., pH 7.5) with a buffering reagent selected from Tris, HEPES, MOPS, acetate, citrate, and phosphate. In some embodiments, sodium acetate or sodium citrate is included as a buffering reagent. In a specific embodiment, the reaction buffer includes Tris-HCl to maintain the pH during the tailing reaction. In some embodiments, the buffering reagent is present at a concentration of between about 5 mM and about 100 mM. In some embodiments, the buffering reagent is present at a concentration of between about 10 mM and about 50 mM. In a specific embodiment, the buffering reagent is present at a concentration of about 50 mM.

For example, Tris-HCl at about 50 mM can be used to maintain the pH of the reaction buffer between 7 and 8 (e.g., at about 7.5).

### Tailing Polymerase

In accordance with the present invention, tailing is performed after in vitro transcription (IVT) of an mRNA of interest. Accordingly, there is no co-transcriptional tailing during IVT. Untailed IVT mRNA is added to a reaction buffer, or vice versa, in accordance with the present invention. A tailing polymerase is added to initiate the tailing reaction.

In some embodiments, the tailing polymerase in the tailing reaction mixture has a concentration of about 20 mg/g to about 75 mg/g. In some embodiments, the tailing polymerase in the reaction mixture has a concentration of about 20 mg/g to 45 mg/g (e.g., 20, 21, 22, 23, 24, 25, 26, 27, 28, 29, 30, 31, 32, 33, 34, 35, 36, 37, 38, 39, 40, 41, 42, 43, 44, or 45 mg/g). In some embodiments, the tailing polymerase in the reaction mixture has a concentration of about 25 mg/g to about 35 mg/g. At the same mass concentration, a reaction mixture comprising a shorter mRNA includes a larger number of molecules than a mixture comprising a longer mRNA. Accordingly, in some embodiments, the concentration of the tailing polymerase in the reaction mixture is adjusted in accordance with the length of the IVT mRNA that is to be tailed. For example, a concentration of tailing polymerase from about 40 mg/g to about 75 mg/g may be appropriate for IVT mRNAs having a length of up to 1 kb. For an IVT mRNA having a length of 1-2 kb, the concentration may be from about 30 mg/g to about 40 mg/g. For an IVT mRNA having a length of 2-4 kb, the concentration may be from about 20 mg/g to about 30 mg/g.

In some embodiments, the tailing polymerase is a polyA polymerase. In some embodiments, the polyA polymerase is a bacterial polyA polymerase or a yeast polyA polymerase. In some embodiments, the bacterial polyA polymerase is an Escherichia coli polyA polymerase.

In some embodiments, the tailing polymerase is a polyC polymerase.

### Divalent Cation

In some embodiments, the reaction buffer comprises a divalent cation. The presence of divalent cation may maintain the activity of the polymerase during tailing.

In some embodiments, the divalent cation is present at a concentration of about 1 mM to about 20 mM. In some embodiments, the divalent cation is at a concentration of between about 5 mM and about 20 mM. The inventors found a divalent cation concentration of 5 mM or more particularly effective in achieving high tailing efficiencies. In some embodiments, the divalent cation is at a concentration of about 5 mM, about 6 mM, about 7 mM, about 8 mM, about 9 mM, or about 10 mM.

In one specific embodiment, the divalent cation is at a concentration of about 5 mM. In another specific embodiment, the divalent cation is at a concentration of about 10 mM. In yet another specific embodiment, the divalent cation is at a concentration of about 15 mM. In a further specific embodiment, the divalent cation is at a concentration of about 20 mM.

In some embodiments, the divalent cation is selected from Mg²⁺ and Mn²⁺. The choice of a particular divalent cation in the reaction buffer may depend on the tailing polymerase. For example, polyA polymerases can typically utilize either Mg²⁺ or Mn²⁺ as a co-factor. The use of Mg²⁺ may be more effective in maintaining the activity of E. coli polyA polymerase in the reaction buffer, whereas for yeast polyA polymerase Mn²⁺ may be more effective.

The concentration of the divalent cation may be varied depending on the particular tailing polymerase and desired tail length. For example, a concentration of about 10 mM Mg²⁺ has been found to be effective in maintaining the activity of the polymerase during the tailing reaction to provide IVT mRNAs with polyA tails of about 200 nucleotides in length. To achieve longer tails higher concentrations may be required.

The divalent cation is typically added to the reaction buffer in the form of a salt, e.g., MgCl₂ or MnCl₂.

### ATP Concentration

In some embodiments, the tailing reaction comprises ATP at a final concentration from about 0.1 mM to about 10 mM. For example, the final ATP concentration may be about 0.1 mM, about 0.2 mM, about 0.3 mM, about 0.5 mM, about 0.6 mM, about 0.7 mM, about 0.8 mM, about 0.9 mM, about 1 mM, about 2 mM, about 3 mM, about 4 mM, about 5 mM, about 6 mM, about 7 mM, about 8 mM, about 9 mM or about 10 mM. ATP may be included in the reaction buffer. In some embodiments, ATP is added separately to the reaction mixture to initiate the tailing reaction.

At the same mass concentration, a reaction mixture comprising a shorter mRNA includes a larger number of molecules than a reaction mixture comprising a longer mRNA. The shorter the mRNA, the more ATP is required in order to tail substantially all IVT mRNAs in a single reaction. Accordingly, in some embodiments, the molar concentration of ATP is adjusted in accordance with the length of the IVT mRNA. Typically, both the concentration of the tailing polymerase and the molar concentration of ATP in the tailing reaction are adjusted, taking into account the length of the IVT mRNA.

For example, an ATP concentration of about 2 mM may be appropriate for IVT mRNAs having a length of up to 1 kb. For an IVT mRNA having a length of 1-2 kb, the ATP concentrations may be adjusted to about 0.8 mM. For an IVT mRNA having a length of 2-4 kb, the concentration may be adjusted to about 0.4 mM.

### Tail Length

The term “tail length” as used herein relates to the average number of ribonucleotides that are added to an IVT mRNA by a tailing polymerase in the reaction buffer of the invention. The tailing conditions determine the resultant tail length of the IVT mRNA. Altering the concentration of the tailing polymerase, the divalent cation, and/or ATP may increase or decrease the tail length. Similar increasing or decreasing the reaction time may influence the overall length of the tail. For example, increasing the concentration of the tailing polymerase and/or ATP may lead to a longer tail being added to the IVT mRNA.

In one specific embodiment, the tail structure of the mRNA comprises a polyA tail. In another specific embodiment, the tail structure of the mRNA comprises a polyC tail. In some embodiments, the tail structure comprises at least 50 adenosine or cytosine ribonucleotides. In a typical embodiment, the tail structure is approximately 100-500 ribonucleotides in length. For example, a tail length of about 200 nucleotides (e.g., a polyA tail) has been shown to stabilize IVT mRNA in vivo.

A polyA or polyC tail on the 3′ terminus of IVT mRNA typically includes at least 50 adenosine or cytosine ribonucleotides, at least 100 adenosine or cytosine ribonucleotides, at least 150 adenosine or cytosine ribonucleotides, at least 200 adenosine or cytosine ribonucleotides, at least 250 adenosine or cytosine ribonucleotides, at least 300 adenosine or cytosine ribonucleotides, at least 350 adenosine or cytosine ribonucleotides, at least 400 adenosine or cytosine ribonucleotides, at least 450 adenosine or cytosine ribonucleotides, at least 500 adenosine or cytosine ribonucleotides, respectively.

In some embodiments, a tail structure includes combination of polyA and polyC stretches with various lengths described herein. In some embodiments, a tail structure includes at least 50%, 55%, 65%, 70%, 75%, 80%, 85%, 90%, 92%, 94%, 95%, 96%, 97%, 98%, or 99% adenosine ribonucleotides. In some embodiments, a tail structure includes at least 50%, 55%, 65%, 70%, 75%, 80%, 85%, 90%, 92%, 94%, 95%, 96%, 97%, 98%, or 99% cytosine ribonucleotides. More typically, a polyA tail on the 3′ terminus of an IVT mRNA produced in accordance with the invention includes between 100 and 500 adenosine ribonucleotides.

In some embodiments, the polyA tail comprises at least two polyA sequences, which are separated from each other by a nucleotide sequence comprising or consisting of at least 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 1 15, 120, 125, 130, 135, 140, 145, or 150 nucleotides. In some embodiments, the nucleotide sequence does not comprise more than 10, 9, 8, 7, 6, 5, 4, 3, or 2 consecutive adenine nucleotides. In some embodiments, the nucleotide sequence which separates the first and the second polyA sequence comprises from 1 to about 200 nucleotides, from 10 to 90, from 20 to 85, from 30 to 80, from 40 to 80, from 50 to 75, or from 55 to 85 nucleotides, wherein the nucleotide sequence does not comprise more than 10, 9, 8, 7, 6, 5, 4, 3, or 2 consecutive adenine nucleotides.

In some embodiments, a portion of the polyA tail is derived from a template DNA and a portion of the polyA tail is generated by enzymatic polyadenylation, e.g., as described in WO2016/091391, which is incorporated herein by reference.

### Tailing Efficiency

The method of the invention provides tailing conditions that improve the tailing efficiency. The conditions allow the tailing polymerase to be more effective in adding a tail to IVT mRNA comprising a modified ribonucleotide. In some embodiments, the tailing efficiency is improved by at least 5% (e.g., about 10%) relative to control (e.g., a non-optimized reaction buffer comprising 250 mM NaCl and no reducing agent). In some embodiments, tailing efficiency is improved by about 10%, about 20%, about 30%, about 40%, or about 50% relative to a control.

An increase in tailing efficiency usually results in a reduction of the percentage of untailed IVT mRNA in a method for tailing IVT mRNA comprising a modified ribonucleotide. In some embodiments, at least 80% of the IVT mRNA is tailed. More typically, at least 90%, at least 91%, at least 92%, at least 93%, at least 94%, at least 95%, at least 96%, at least 97%, at least 98%, or at least 99%, of the IVT mRNA is tailed. In some embodiments, substantially all of the IVT mRNA is tailed. For example, a preparation in which at least 80% (e.g., at least 90% or 95%) of the IVT mRNA is tailed is acceptable in the manufacturing of an mRNA for therapeutic use. In some embodiments, a method for tailing IVT comprising a modified ribonucleotide in accordance with the invention yields a preparation in which no untailed IVT mRNA is detectable. Untailed mRNA can be detected by capillary gel electrophoresis, as demonstrated in the examples. Alternatively, RNase H digestion coupled with ultrahigh pressure liquid chromatography (UHPLC) separation and liquid chromatography-mass spectrometry (LC-MS) detection can be used to determine the percentage of IVT mRNA that is tailed.

Typically, a reduction in the percentage of untailed IVT mRNA results in the tailed IVT mRNA having an average tail length close to the desired tail length. For example, as exemplified herein, the average tail length may be 45%-50% longer than the desired length when using an unoptimized reaction buffer. In contrast, the inventors observed that the average tail length was at most about 30%-25% longer than the desired tail length when using an optimized reaction buffer of the invention, and more typically only about 5% longer than desired. In one embodiment, the desired tail length is at least 100 nucleotides (e.g., 100-500 nucleotides). For example, the desired tail length may be a particular value (e.g., 200 nucleotides), and the average length of the tailed IVT mRNA may be within about 30%, about 25%, about 20%, about 15%, about 10%, or about 5% of that value.

In some embodiments, the tailing efficiency of the tailing reaction is at least 80%, e.g., at least 80% of the IVT mRNA is tailed and has an average tail length within 20% of the desired length. In some embodiments, the tailing efficiency of the tailing reaction is at least 90%, at least 91%, at least 92%, at least 93%, at least 94%, at least 95%, at least 96%, at least 97%, at least 98%, or at least 99%. For example, at least 90%, at least 91%, at least 92%, at least 93%, at least 94%, at least 95%, at least 96%, at least 97%, at least 98%, or at least 99% of the IVT mRNA is tailed and has an average tail length within 20% of the desired length. A suitable assay for measuring poly A tail length using a minor-groove binding dye and one or more ribonucleases is described in WO 2022/232499.

### Exemplary Tailing Conditions

In particular embodiments, the invention is directed to a method for tailing IVT mRNA comprising a modified ribonucleotide that comprises adding the IVT mRNA to a tailing polymerase in a reaction buffer comprising a buffering agent (e.g., Tris-HCl) at a concentration of about 50 mM, an alkali metal salt (e.g., NaCl or KCl) at a concentration of about 5 mM, divalent cation (e.g., Mg²⁺, for instance MgCl₂) at a concentration of about 10 mM, and a reducing agent (e.g., DTT) at a concentration of about 10 mM. If necessary, the pH of the reaction buffer may be adjusted to about 7.5.

### Optional Capping Step

IVT mRNAs with a methylated 5′ cap structure are efficiently translated in vivo. The IVT process can include a cap analogue which is added co-transcriptionally. Alternatively, the 5′ cap structure can be added enzymatically after the IVT reaction has been completed. At least 90% of IVT mRNA subjected to enzymatic capping can comprise Cap1 structures.

Several types of 5′ caps are known. A 7-methylguanosine cap (also referred to as “m7G” or “Cap 0”), comprises a guanosine that is linked through a 5′-5′-triphosphate bond to the first transcribed nucleotide. A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp, (5′(A,G(5′)ppp(5′)A, and G(5′)ppp(5′)G. Additional cap structures are described in U.S. Publication Nos. US 2016/0032356 and US 2018/0125989, which are incorporated herein by reference.

During co-transcriptional capping, a cap analogue is included in the IVT reaction mixture. The cap analogue can be incorporated as the first “base” in a nascent RNA strand. The cap analogue may be Cap 0, Cap 1, Cap 2, m⁶Am, or a chemical cap analogue. For example, the following chemical cap analogues may be used to generate the 5′-guanosine cap structure according to the manufacturer's instructions: 3′-O-Me-m7G(5′)ppp(5′)G (the ARCA cap); G(5′)ppp(5′)A; G(5′)ppp(5′)G; m7G(5′)ppp(5′)A; m7G(5′)ppp(5′)G; m7G(5′)ppp(5′)(2′OMeA)-pG; m7G(5′)ppp(5′)(2′OMeA)pU; m7G(5′)ppp(5′)(2′OMeG)pG (New England BioLabs, Ipswich, MA; TriLink Biotechnologies).

A vaccinia virus capping enzyme may be used to generate the Cap 0 structure: m7G(5′)ppp(5′)G. A Cap 1 structure may be generated using both vaccinia virus capping enzyme and a 2′-O methyl-transferase to generate: m7G(5′)ppp(5′)G-2′-O-methyl. A Cap 2 structure may be generated from the Cap 1 structure followed by the 2′-O-methylation of the 5′-antepenultimate nucleotide using a 2′-O methyltransferase. A Cap 3 structure may be generated from the Cap 2 structure followed by the 2′-O-methylation of the 5′-preantepenultimate nucleotide using a 2′-O methyltransferase.

In some embodiments, a method in accordance with the invention further comprises a step of capping the IVT mRNA. The capping step may involve adding a capping enzyme (guanylyltransferase) and a guanine. A suitable capping enzyme may be derived from a Vaccinia virus (Vaccine virus guanylyltransferase). Typically, the capping step also comprises adding a guanine methyltransferase and a 2′-O-methyltransferase. Capping may be performed separately, e.g., after in vitro transcription. The capping step is commonly performed prior to the tailing of the IVT mRNA.

In some embodiments, the reaction buffer of the invention is added after the IVT mRNA has been capped to adjust the reaction conditions prior to the addition of the tailing polymerase. Reactants that may form part of the capping buffer (e.g., reducing agents and/or divalent cations) are typically consumed during the capping reaction. Addition of the reaction buffer of the invention provides components such as a reducing agent at a concentration suitable for the tailing reaction.

In a specific embodiment, the IVT mRNA may comprise a 5′ cap with the following structure:

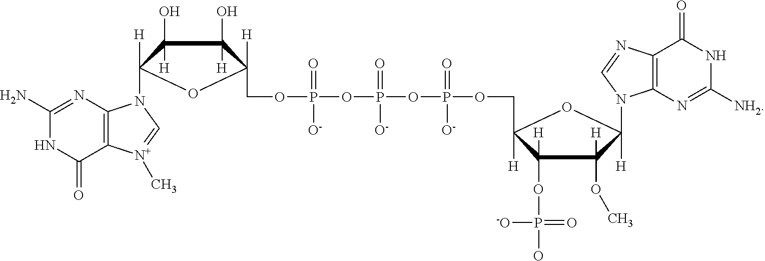

### Purification

In some embodiments, the IVT mRNA is purified before it is tailed in accordance with the invention. In some embodiments, the IVT mRNA is purified after tailing. In some embodiments, the IVT mRNA is capped prior to adding the tail. In some embodiments, the capped IVT mRNA is purified prior to tailing.

Various methods may be used to purify mRNA before or after capping and/or tailing. In some embodiments, the mRNA is purified by precipitation and centrifugation. In some embodiments, the mRNA is purified by filtration using, e.g., Normal Flow Filtration or Tangential Flow Filtration (TFF).

Suitable purification methods include those described in published U.S. Application Nos. US 2016/0040154, US 2015/0376220, US 2018/0251755, US 2018/0251754, US 2020/0095571, US 2021/0388338, and US 2021/0002635, and in U.S. provisional application No. 63/086,095 filed on Oct. 1, 2020, all of which are incorporated by reference herein and may be used to practice the present invention.

### Reaction Buffer

The invention also relates to a reaction buffer comprising 30 mM or less of an alkali metal salt and 5 mM or more of a reducing agent at 1× concentration. As described herein, such a reaction buffer has been found to be particularly suitable for use in a method for tailing IVT mRNA comprising a modified ribonucleotide. Although the buffer was specifically developed to address reduced efficiency observed when tailing IVT mRNA comprising a modified ribonucleotide in a prior art reaction buffer, the inventors found that the reaction buffer of the invention can also be used for tailing IVT mRNA that does not comprise a modified ribonucleotide.

The following paragraphs describe the composition of the 1× reaction buffer in greater detail. It is generally understood by the skilled person that reaction buffers are typically provided at 10× concentration, e.g., for shipping and storage. Invention expressly includes embodiments of the reaction buffer at 10× concentration, e.g., for shipping and storage. For example, as a 10× stock solution, the components of the reaction buffer would be 10× more concentrated, e.g., the stock solution at 10× would comprise 300 mM or less of an alkali metal salt and 50 mM or more of a reducing agent, etc.

### Alkali Metal Salt

In some embodiments, the alkali metal salt in the reaction buffer has a concentration of about 1 mM to about 30 mM or of about 5 mM to about 30 mM, e.g., about 5 mM to about 27.5 mM. In some embodiments, the alkali metal salt in the reaction buffer has a concentration of about 25 mM or less, e.g., about 1 mM to about 25 mM or about 5 mM to about 25 mM. In some embodiments, the alkali metal salt in the reaction buffer has a concentration of about 20 mM or less, e.g., about 1 mM to about 20 mM or about 5 mM to about 20 mM. In some embodiments, the alkali metal salt in the reaction buffer has a concentration of about 15 mM or less, e.g., about 1 mM to about 15 mM or about 5 mM to about 15 mM. In some embodiments, the alkali metal salt in the reaction buffer has a concentration of about 10 mM or less, e.g., about 1 mM to about 10 mM or about 5 mM to about 10 mM.

In particular embodiments, the alkali metal salt in the reaction buffer has a concentration of about 5 mM or less, e.g., about 1 mM to about 5 mM. In specific embodiments, the alkali metal salt has a concentration of about 5 mM. In other embodiments, the alkali metal salt has a concentration of about 4 mM, about 3 mM, about 2 mM, or about 1 mM.

In some embodiments, the alkali metal salt in the reaction buffer is NaCl. In some embodiments, the alkali metal salt in the reaction buffer is KCl.

In some embodiments, the alkali metal salt in the reaction buffer is NaCl and has a concentration of about 30 mM or less (e.g., 25 mM, 20 mM, 15 mM, or 10 mM). In some embodiments, the alkali metal salt in the reaction buffer is NaCl and has a concentration of about 5 mM to about 30 mM.

In particular embodiments, the alkali metal salt in the reaction buffer is NaCl and has a concentration of about 5 mM or less, e.g., about 1 mM to about 5 mM, such as about 4 mM, about 3 mM, or about 2 mM. In a specific embodiment, the alkali metal salt in the reaction buffer is NaCl and has a concentration of about 5 mM.

In some embodiments, the alkali metal salt in the reaction buffer is KCl and has a concentration of about 30 mM or less (e.g., between about 1 mM and about 30 mM such as about 25 mM, 20 mM, 15 mM, or 10 mM). In some embodiments, the alkali metal salt in the reaction buffer is KCl and has a concentration of about 5 mM to about 30 mM.

In particular embodiments, the alkali metal salt in the reaction buffer is KCl and has a concentration of about 5 mM or less, e.g., about 1 mM to about 5 mM, such as about 4 mM, about 3 mM, or about 2 mM. In a specific embodiment, the alkali metal salt in the reaction buffer is KCl and has a concentration of about 5 mM.

### Reducing Agent

In some embodiments, the reducing agent has a concentration of about 5 mM or more, e.g., about 5 mM to about 50 mM. In some embodiments, the reducing agent has a concentration of about 5 mM to about 20 mM. In some embodiments, the reducing agent has a concentration of about 5 mM, about 10 mM, about 15 mM, or about 20 mM. In specific embodiments, the reducing agent has a concentration of about 10 mM.

In some embodiments, the reducing agent reduces disulfide bonds. Suitable reducing agent include dithiothreitol (DTT), 2-mercaptoethanol (2-ME), and tris(2-carboxyethyl)phosphine (TCEP).

In a specific embodiment, the reducing agent is DTT. In some embodiments, the reaction buffer comprises DTT at a concentration of at least 5 mM, e.g., about 5 mM to about 50 mM. In a specific embodiment, the reaction buffer comprises DTT at a concentration of about 10 mM.

pH

In some embodiments, the reaction buffer has a pH of between about 6 and about 8.5. In some embodiments, the reaction buffer has a pH of between about 7 and pH 8 (e.g., between about 7.2 and 7.8). In some embodiments, the reaction buffer has a pH of about 7.0, about 7.1, about 7.2, about 7.3, about 7.4, about 7.5, about 7.6, about 7.7, about 7.8, about 7.9, or about 8.0. In a specific embodiment, the reaction buffer has a pH of about 7.5.

In some embodiments, the reaction buffer comprises Tris, HEPES, MOPS, acetate, citrate, or phosphate as a buffering reagent. In some embodiments, the reaction buffer comprises sodium acetate or sodium citrate as a buffering reagent. In a specific embodiment, the reaction buffer includes Tris-HCl as a buffering reagent. In some embodiments, the buffering reagent is present at a concentration of between about 5 mM and about 100 mM. In some embodiments, the buffering reagent is present at a concentration of between about 10 mM and about 50 mM. In a specific embodiment, the buffering reagent is present at a concentration of about 50 mM.

For example, Tris-HCl at about 50 mM can be used to maintain the pH of the reaction buffer between 7 and 8 (e.g., at about 7.5).

### Divalent Cation

In some embodiments, the reaction buffer comprises a divalent cation. In some embodiments, the divalent cation is selected from Mg²⁺ and Mn²⁺. In some embodiments, the reaction buffer comprises MgCl₂ or MnCl₂.

In some embodiments, the divalent cation is present at a concentration of between about 1 mM and about 20 mM. In some embodiments, the divalent cation is at a concentration of between about 5 mM and about 20 mM. The inventors found a divalent cation concentration of 5 mM or more particularly effective in achieving high tailing efficiencies. In some embodiments, the divalent cation is at a concentration of about 1 mM, about 2 mM, about 3 mM, about 4 mM, about 5 mM, about 6 mM, about 7 mM, about 8 mM, about 9 mM, or about 10 mM.

In one specific embodiment, the divalent cation is at a concentration of about 5 mM. In another specific embodiment, the divalent cation is at a concentration of about 10 mM. In yet another specific embodiment, the divalent cation is at a concentration of about 15 mM. In a further specific embodiment, the divalent cation is at a concentration of about 20 mM.

The choice and concentration of the divalent cation may be varied, e.g., depending on the particular tailing polymerase with which the buffer is to be used. For example, a concentration of 5-20 mM Mg²⁺ (e.g., about 10 mM Mg²⁺) has been found to be effective in maintaining the activity of an E. coli poly A polymerase during tailing reactions described herein.

### Exemplary Reaction Buffers

An exemplary reaction buffer for use in the methods described herein comprises a buffering reagent (e.g., Tris-HCl) at a concentration of about 50 mM, an alkali metal salt (e.g., NaCl or KCl) at a concentration of about 5 mM, a divalent cation (e.g., Mg²⁺, for instance MgCl₂) at a concentration of about 10 mM, and a reducing agent (e.g., DTT) at a concentration of about 10 mM. If necessary, the pH of the reaction buffer may be adjusted to about 7.5.

For example, a reaction buffer of the invention may have the composition shown in Table 1 below. In a typical embodiment, the pH of the buffer is about 7.5.

TABLE 1

| Component | Concentration 1x/10x |
|---|---|
| Tris-HCl | 50 mM/500 mM |
| NaCl | 5 mM/50 mM |
| MgCl₂ | 10 mM/100 mM |
| DTT | 10 mM/100 mM |

The reaction buffer is used for tailing IVT mRNA at a 1× concentration. The reaction buffer can be shipped or stored at a 10× concentration.

Methods of Manufacturing mRNA

The invention also relates to methods of manufacturing mRNA that comprise synthesizing mRNA by in vitro transcription; and tailing the in vitro transcribed (IVT) mRNA using a method described herein, namely by adding the IVT mRNA to a tailing polymerase in a reaction buffer comprising 30 mM or less of an alkali metal salt and 5 mM or more of a reducing agent. Typically, the IVT mRNA comprises a modified ribonucleotide (e.g., N1-methylpseudouridine).

In some embodiments, the mRNA is synthesized in batches. The inventors have used the tailing methods described herein to tail batches of 2 mg and 5 mg IVT mRNA. The inventors found that corresponding conditions can be used to tail batches of about 1 g and about 16 g of IVT mRNA. Accordingly, in some embodiments, a batch comprises at least 1 mg of IVT mRNA (e.g., 2 mg, 3 mg, 4 mg, 5 mg, 6 mg, 7 mg, 8 mg, or 9 mg). In some embodiments, a batch comprises at least 10 mg of IVT mRNA (e.g., 11 mg, 12 mg, 13 mg, 14 mg, 15 mg, 16 mg, 17 g, 18 g, 19 g, or 20 g). In some embodiments, a batch comprises at least 100 mg of IVT mRNA (e.g., 200 mg, 300 mg, 400 mg, 500 mg, 600 mg, 700 mg, 800 mg, or 900 mg). In other embodiments, a batch comprises at least 1 g of IVT mRNA (e.g., 5 g, 10 g, 15 g, 20 g, or 25 g). In further embodiments, a batch comprises at least 50 g of IVT mRNA (e.g., 75 g, 100 g, 150 g, 200 g, or 250 g).

In other embodiments, a batch comprises at least 0.5 kg of IVT mRNA (e.g., 0.75 kg, 1 kg, or 5 kg). In some embodiments, 10 kg, 50 kg, 100 kg, 1000 kg, or more of IVT mRNA is synthesized in a single batch and then tailed in accordance with the methods of the invention.

The inventors surprisingly found that the presence of a modified ribonucleotide at the terminal 3′ residue of IVT mRNA can reduce the tailing efficiency dramatically. Accordingly, the invention also relates to a method for generating (or manufacturing) an in vitro transcribed (IVT) messenger RNA (mRNA) comprising a modified ribonucleotide, wherein the method comprises: (i) preparing a DNA template wherein the terminal 3′ residue of the DNA template does not encode a modified ribonucleotide of the IVT mRNA; and (ii) transcribing the DNA template in an in vitro transcription reaction comprising the modified ribonucleotide. As the DNA template is prepared in such a manner that the terminal 3′ residue of the DNA template does not encode a modified ribonucleotide, the IVT mRNA obtained in step (ii) does not include a modified ribonucleotide as the 3′ terminal residue and can be tailed more efficiently.

Compositions Comprising Tailed IVT mRNA

The invention also relates to compositions comprising tailed IVT mRNAs obtainable by the methods described herein. In some embodiments, at least 80% of the IVT mRNA is tailed. More typically, at least 90%, at least 91%, at least 92%, at least 93%, at least 94%, at least 95%, at least 96%, at least 97%, at least 98%, or at least 99%, of the IVT mRNA is tailed. For example, a preparation in which at least 80% of the IVT mRNA is tailed is acceptable in the manufacturing of an mRNA for therapeutic use.

In some embodiments, substantially all of the IVT mRNA is tailed in compositions obtainable by the tailing methods of the invention. Accordingly, compositions of the invention comprising tailed IVT mRNAs are characterized by the absence of detectable amounts of untailed IVT mRNA. A suitable method for determining the absence of untailed IVT mRNA is capillary gel electrophoresis. As demonstrated in the examples, the methods of tailing IVT mRNA described herein result in a composition comprising tailed IVT mRNAs that is characterized by the absence of a peak corresponding to untailed IVT mRNA, when analyzed by capillary gel electrophoresis.

Moreover, the compositions are typically characterized by a narrow size distribution of the tailed IVT mRNAs. A reduction in the percentage of untailed IVT mRNA commonly results in the tailed IVT mRNA having an average tail length close to the desired tail length. For example, a desired tail length may be 100 to 500 nucleotides, and at least 80% of the IVT mRNA in the composition has the desired tail length. In some embodiments, the desired tail length is at least 100 nucleotides, and at least 80% (e.g., at least 90% or at least 95%) of the IVT mRNA in the composition has the desired tail length. In some embodiments, the desired tail length is at least 150 nucleotides, and at least 80% (e.g., at least 90% or at least 95%) of the IVT mRNA in the composition has the desired tail length. In some embodiments, the desired tail length is about 200 nucleotides, and at least 80% (e.g., at least 90% or at least 95%) of the IVT mRNA in the composition has the desired tail length. In some embodiments, the desired tail length is about 250 nucleotides, and at least 80% (e.g., at least 90% or at least 95%) of the IVT mRNA in the composition has the desired tail length. In some embodiments, the desired tail length is about 500 nucleotides, and at least 80% (e.g., at least 90% or at least 95%) of the IVT mRNA in the composition has the desired tail length.

### EXAMPLES

The following examples are included for illustrative purposes only and are not intended to limit the scope of the invention.

### Example 1. Preparing In Vitro Transcribed mRNA

In vitro transcribed (IVT) mRNA was prepared as described in Example 1 of WO 2021/168052, which is incorporated herein by reference. Briefly, for each gram of mRNA transcribed, a reaction containing a linearized double-stranded DNA plasmid with an RNA polymerase-specific promoter, RNA polymerase (e.g., SP6 polymerase or T7 polymerase), RNase inhibitor, pyrophosphatase, NTPs, DTT, and a buffering reagent was prepared with RNase-free water. The reaction mixture was then incubated at 37° C. for 60 to 90 min. In Examples 2-5, UTP was replaced with N1-methylpseudouridine triphosphate to prepare IVT mRNA comprising a modified ribonucleotide. DNase I was added to stop the reaction, and the reaction mixture was incubated for an additional 15 minutes at 37° C. The resulting IVT mRNA was purified.

Purified IVT mRNA was then capped by mixing it with GTP (1.0 mM), S-adenosyl methionine, RNase inhibitor, 2′-O-Methyltransferase, and a guanylyl transferase in a suitable reaction buffer (e.g., a 10× buffer comprising 500 mM Tris-HCl (pH 8.0), 60 mM KCl, 12.5 mM MgCl₂). The resulting reaction mixture was incubated at 37° C. for 30 to 90 minutes.

PolyA tailing is typically performed by adding PolyA polymerase, ATP, and tailing reaction buffer (10×: 500 mM Tris-HCl (pH 7.5), 2.5 M NaCl, 100 mM MgCl₂) to the reaction and incubating the reaction mixture at 37° C. for 20 to 60 minutes.

### Example 2. Optimizing Reaction Buffer for Tailing

It was observed that tailing efficiency was reduced when mRNA molecules included a modified ribonucleotide, such as N1-methylpseudouridine, as compared to the tailing efficiency that was obtained for mRNA molecules that did not comprise a modified ribonucleotide. This example illustrates that optimizing the concentration of an alkali metal salt in the reaction buffer used for tailing in vitro transcribed (IVT) mRNA comprising a modified ribonucleotide can improve tailing efficiency.

To test this hypothesis, reaction buffers comprising different concentrations of a buffering reagent and an alkali metal salt were used for tailing IVT mRNA with a tailing polymerase. A polyA tailing polymerase and ATP were added to each of the test reaction buffers. The IVT mRNA was prepared as described in Example 1 and included a modified ribonucleotide (N1-methylpseudouridine). The reaction conditions were chosen to result in a target tail length of about 500 ribonucleotides.

The tested conditions and resulting tailing efficiencies and tail lengths are summarized in Table 2. The listed molar concentrations of the buffering reagent, alkali metal salt, and divalent cation are the concentrations found in the 1× reaction buffer.

TABLE 2

| Buffer | Tris | NaCl | MgCl₂ | % Untailed- | % Main- | Tail Length |
|---|---|---|---|---|---|---|
| (1x) | (mM) | (mM) | (mM) | peak | peak | (nt) |
| 1 | 5 | 5 | 10 | 1.7 | 91.9 | 241 |
| 2 | 50 | 20.5 | 10 | 1.7 | 91.3 | 450 |
| 3 | 27.5 | 27.5 | 10 | 2 | 91.8 | 413 |
| 4 | 27.5 | 50 | 10 | 1.9 | 92.7 | 512 |
| 5 | 5 | 27.5 | 10 | 1.7 | 93.5 | 327 |
| 6 | 50 | 50 | 10 | 2 | 93.1 | 598 |
| 7 | 27.5 | 27.5 | 10 | 1.6 | 90.8 | 426 |
| 8 | 50 | 5 | 10 | 1.4 | 91.2 | 450 |
| 9 | 27.5 | 5 | 10 | 1.8 | 90.9 | 376 |
| 10 | 27.5 | 27.5 | 10 | 1.6 | 90.5 | 438 |
| 11 | 50 | 35.2 | 10 | 1.7 | 90.4 | 561 |
| 12 | 5 | 50 | 10 | 1.9 | 90.5 | 401 |
| Control | 50 | 250 | 10 | 8.2 | 84.8 | 1214 |

Buffers 3, 7, and 10 were internal replicates to determine run variability. As a control, a reaction buffer was included that had previously proven effective in tailing reactions with IVT mRNA that did not include a modified ribonucleotide. This buffer was previously described in WO 2021/168052. At 10×, it is composed of 500 mM Tris-HCl as the buffering reagent, 2.5 M of the alkali metal salt NaCl, and 100 mM MgCl₂. The pH of the control buffer was 7.5.

Capillary gel electrophoresis was used to evaluate tailing of mRNA. Briefly, the standard sensitivity RNA analysis kit (15 nt) was purchased from Agilent and used in capillary electrophoresis runs on the Fragment Analyzer instrument with a twelve-capillary array (Agilent). Upon gel priming, 300 ng of total RNA was mixed with diluent marker at 1:11 (RNA:Marker) ratio and 24 μL was loaded per well in a 96-well plate. The molecular weight indicator ladder was prepared by mixing 2 μl of the standard sensitivity RNA ladder with 22 μl diluent marker. Sample injection was at 5.0 kV, 4 seconds and sample separation at 8.0 kV, 60.0 min. Electropherogram of each sample was processed through the ProSize 2 software (Advanced Analytical), producing tabulated sizes (nt) and abundances of fragments present in the sample.

As can be seen from Table 2, about 8% of the IVT mRNA was untailed when the control buffer with 250 mM NaCl was used to tail an IVT mRNA comprising a modified ribonucleotide. Notably, the average tail length of about 1200 ribonucleotides far exceeded the target tail length of 500 ribonucleotides. All 12 test conditions improved tailing efficiency at least 4-fold relative to the control buffer. A key parameter appeared to be the alkali metal salt of the reaction as a 7- to 8-fold reduction of the alkali metal salt concentration to about 30 mM improved the tailing efficiency relative to the reaction buffer that was used as a control. Tailing efficiency was improved more than 5-fold when the concentration of the alkali metal salt was reduced 50-fold to 5 mM, while keeping the concentration of the buffering reagent unchanged at 50 mM—cf. control buffer and buffer 8 in Table 2. Only 1.4% of the IVT mRNA remained untailed when buffer 8 was used. Moreover, under these conditions, when tailing occurred, an average tail length of 450 ribonucleotides was achieved.

The overall reduction of the reaction buffer's ionic strength resulted in a significant reduction of untailed IVT mRNA. However, in some instances, this reduction was accompanied by a decrease in the average tail length. For example, reducing the concentration of the buffering reagent 10-fold in addition to a 50-fold reduction of the alkali metal salt concentration did not provide an additional benefit—cf. buffer 1 and buffer 8 in Table 2. At 241 ribonucleotides, the average tail length was less than half of the desired target length of 500 ribonucleotides when buffer 1 was used as the reaction buffer.

This example demonstrates that optimizing the concentration of the alkali metal salt in the reaction buffer used for tailing IVT mRNA comprising a modified ribonucleotide to 30 mM or less (e.g., 5 mM or less) improves the efficiency of the tailing reaction. Given that performing the tailing reaction with buffer 8 resulted at the lowest percentage of untailed IVT mRNA and an average tail length close to the desired target length, this buffer composition was used as the basis for subsequent experiments.

### Example 3. Addition of a Reducing Agent

This example illustrates that tailing efficiency can be further improved by the addition of a reducing agent to a reaction buffer comprising 30 mM or less of an alkali metal salt.

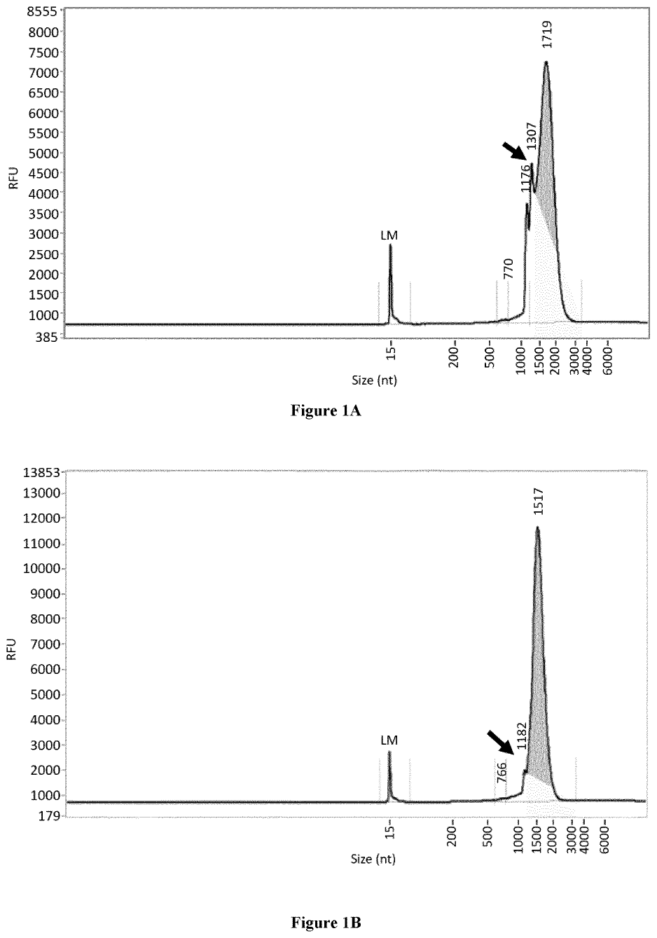

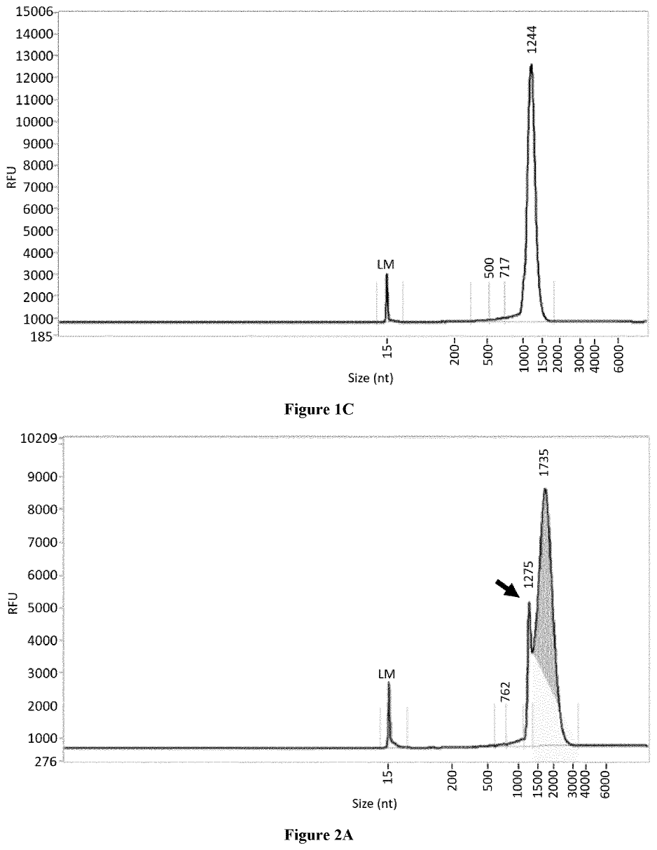

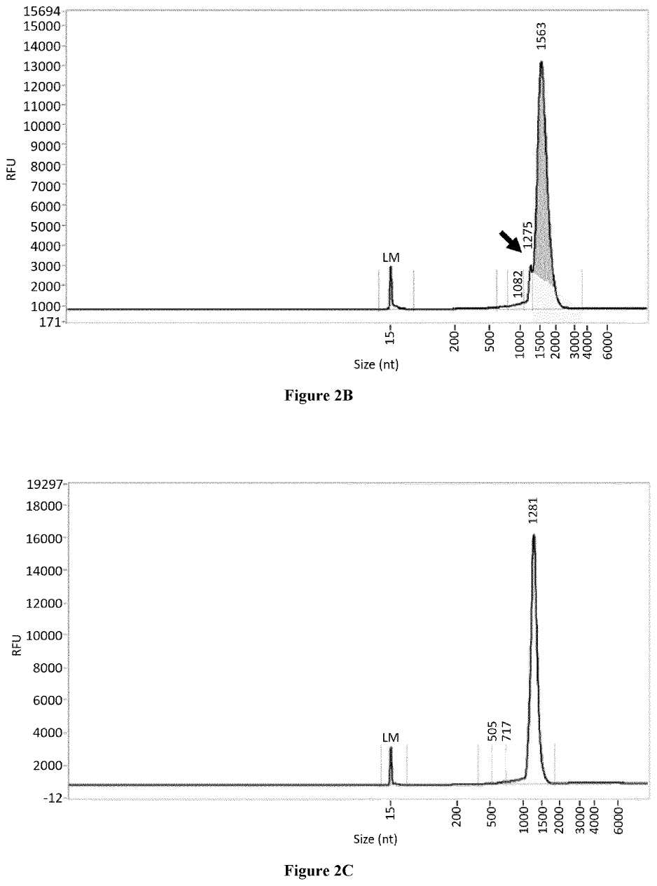

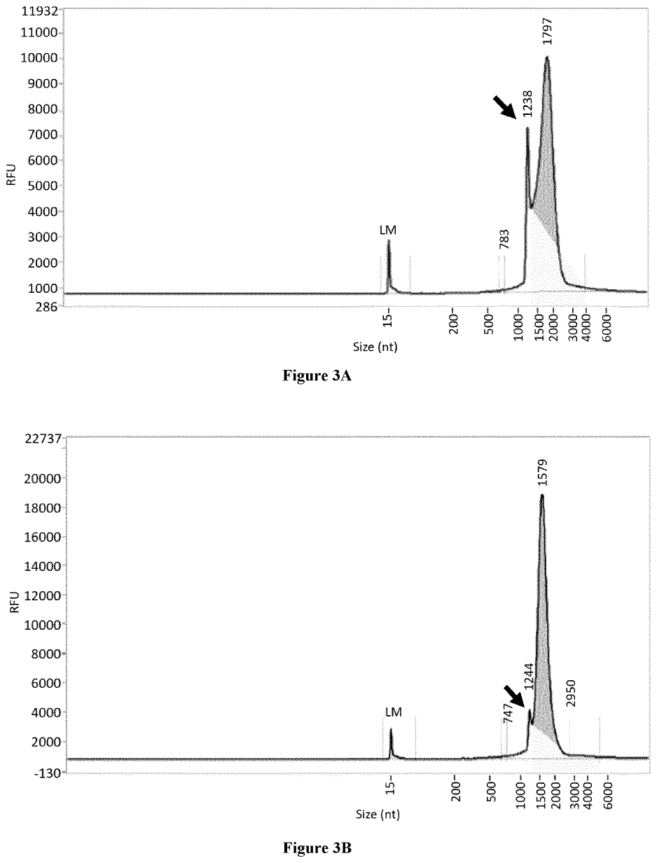

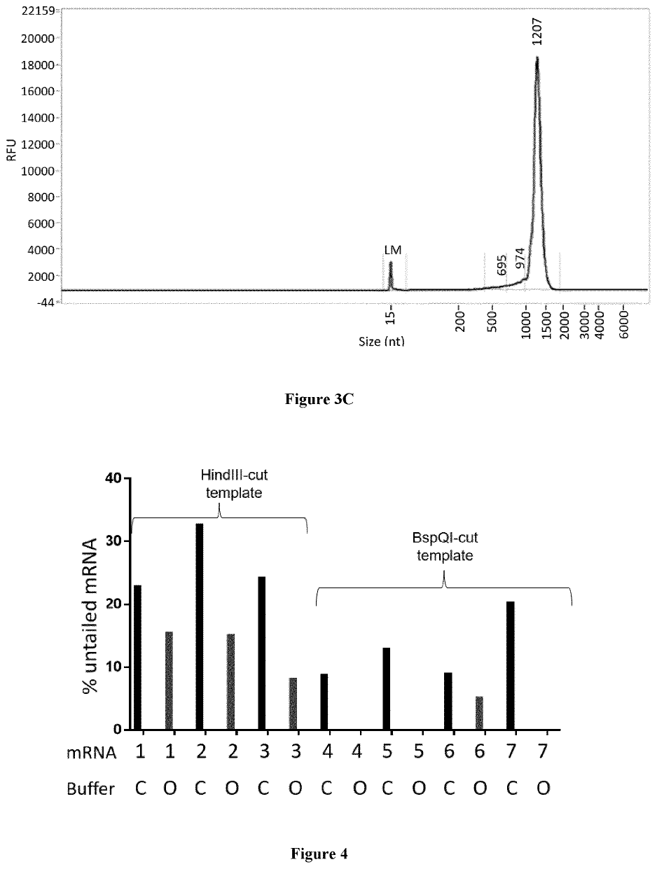

Three different IVT mRNAs (mRNA A composed of 1256 ribonucleotides, mRNA B composed of 1268 ribonucleotides, and mRNA C composed of 1205 ribonucleotides), each comprising a modified ribonucleotide (N1-methylpseudouridine), were tailed in the control buffer or buffer 8, as described in Example 2 (see Table 2). In addition, each IVT mRNA was also tailed in a modified version of buffer 8, which further included a reducing agent (DTT). The target tail length was 200 nucleotides. The resulting tailed IVT mRNA was analyzed by capillary gel electrophoresis. The results obtained for mRNA A, mRNA B, and mRNA C are summarized in FIGS. 1, 2, and 3, respectively. For each IVT mRNA, use of the control buffer resulted in a clearly identifiable additional peak preceding the main peak, indicating a significant amount of untailed IVT mRNA (see FIGS. 1A, 2A, and 3A). As observed in Example 2, use of buffer 8 markedly reduced the percentage of untailed IVT mRNA compared to the control buffer, as can be seen from the dramatic reduction in size of the additional peak preceding the main peak (see FIGS. 1B, 2B, and 3B). When a reducing agent was added to buffer 8, the additional peak was no longer discernible, indicating that the amount of untailed IVT mRNA was further reduced (see FIGS. 1C, 2C, and 3C).

Notably, despite using a relatively high concentration of the reducing agent in buffer 8 (10 mM at 1×), the performance of the polyA polymerase was not negatively impacted. This is surprising because, while commercially available reaction buffers sometimes contain a reducing agent, it is typically present at a much lower concentration (e.g., 0.2 mM at 1×).

Moreover, the improvement resulting from a reduced alkali metal salt concentration in the reaction buffer and the further increased tailing efficiency achieved by adding a reducing agent to the reaction buffer were observed independent of the ribonucleotide sequence of the IVT mRNA. Indeed, similar improvements were observed for each of mRNA A, mRNA B, and mRNA C.

This example demonstrates that the tailing efficiency can be further improved by adding a reducing agent to a reaction buffer comprising a reduced amount of alkali metal salt as compared to the control buffer. The observed improvement is independent of the ribonucleotide sequence of the IVT mRNA.

### Example 4. Concentration of the Reducing Agent

This example demonstrates that tailing efficiency can be improved by adding 5 mM or more of a reducing agent to a reaction buffer comprising 5 mM or less of an alkali metal salt.

To determine a suitable concentration range for the reducing agent, the experiment described in Example 3 was repeated with varying concentrations of DTT as the reducing agent in buffer 8. The following concentrations were tested: 0 mM, 5 mM, 10 mM, 15 mM, 25 mM, and 50 mM. The resulting tailed IVT mRNA was analyzed by capillary gel electrophoresis. The results are summarized in Table 3.

TABLE 3

| DTT concentration | Untailed mRNA detected |
|---|---|
| 0 mM | Yes |
| 5 mM | No |
| 10 mM | No |
| 15 mM | No |
| 25 mM | No |
| 50 mM | No |

At 0 mM, an additional peak preceding the main peak was observed, indicating a significant amount of untailed IVT mRNA. When a reducing agent was added to buffer 8, the additional peak was no longer discernible, consistent with previous observation. No additional peak was observed at reducing agent concentrations ranging from 5-50 mM.

This example demonstrates that tailing efficiency can be improved adding 5 mM or more of a reducing agent to a reaction buffer comprising 5 mM or less of an alkali metal salt.

### Example 5. Improved Tailing Efficiency without Modified Ribonucleotide at 3′ End

This example illustrates that tailing is improved with the optimized reaction buffer (buffer 8 with DTT) identified in Example 3, even when a modified ribonucleotide is present as the terminal ribonucleotide at the 3′ end of the IVT mRNA. Tailing efficiency is highest when the IVT mRNA does not include a modified ribonucleotide at the 3′ end. This example therefore also demonstrates that tailing IVT mRNA comprising a modified ribonucleotide can be improved by providing untailed IVT mRNA that does not comprise the modified ribonucleotide at the 3′ end.

Two different plasmid backbones (I) and (II) were prepared. Different template nucleic acids were inserted into the two backbones to assess the impact of the ribonucleotide sequence on the tailing efficiency of the IVT mRNA. Template-containing plasmids with backbone (I) were linearized with HindIII, and template-containing plasmids with backbone (II) were linearized with BspQI. IVT reactions including linearized template DNA were performed as described in Example 1. A modified uridine (N1-methylpseudouridine) was included in the reaction mixture in place of UTP. HindIII-cut templates yielded IVT mRNA with the modified ribonucleotide as the ultimate ribonucleotide at the 3′ end. BspQI-cut templates yielded IVT mRNAs that did not include a modified ribonucleotide at the 3′ end. The resulting IVT mRNAs were tailed either in the control buffer described in Example 2, or in buffer 8 additionally comprising 10 mM DTT as described in Example 3.

As can be seen from FIG. 4, tailing efficiency was reduced for IVT mRNA synthesized from HindIII-cut templates comprising a modified ribonucleotide at the 3′ end, when compared to mRNA generated from BspQ1-cut templates.

Surprisingly, even in the presence of a modified uridine at the 3′ end, only 10%-15% of the IVT mRNAs remained untailed when buffer 8 with 10 mM DTT was used in the tailing reaction. This compared favorably to results obtained with the control buffer (compare HindIII-cut mRNA tailed in the control buffer (buffer ‘C’) to corresponding mRNA tailed in buffer 8 with 10 mM DTT (buffer ‘O’) in FIG. 4). Generally, using the control buffer described in Example 2, tailing efficiency was about two-fold lower. In some instances, more than 30% of the IVT mRNA with a modified ribonucleotide at the 3′ end remained untailed when using the control buffer. Unlike in Example 3, overall tailing efficiency varied depending on the ribonucleotide sequence of the IVT mRNA. Sequence optimization may further improve tailing efficiency.

Notably, when the IVT mRNA was prepared from a BspQI-cut template, and therefore did not have a modified ribonucleotide at the 3′ end, the use of the control buffer caused up to 20% of the IVT mRNA to remain untailed. In contrast, even with the most challenging ribonucleotide sequence, untailed IVT mRNA did not exceed 5% when buffer 8 with 10 mM DTT was used. In fact, when buffer 8 with 10 mM DTT was used, no untailed IVT mRNA was detected in 3 out of 4 test samples.

This example demonstrates that an optimized reaction buffer comprising 5 mM or less of an alkali metal salt and 5 mM or more of a reducing agent improves tailing efficiency, independent of the ribonucleotide at the 3′ end of the IVT mRNA or the mRNA ribonucleotide sequence. Tailing efficiency was highest when the IVT mRNA did not include a modified ribonucleotide at the 3′ end. Therefore, this example also demonstrates that tailing IVT mRNA comprising a modified ribonucleotide can be improved by providing untailed IVT mRNA that does not comprise the modified ribonucleotide at the 3′ end. Interestingly, in comparison to an unoptimized reaction buffer, the optimized reaction buffer (buffer 8 with DTT) even improved the tailing efficiency of IVT mRNA with a modified ribonucleotide at the 3′ end.

### Example 6. Use of Optimized Reaction Buffer with Unmodified IVT mRNA

This example demonstrates that a reaction buffer that has been optimized for tailing in vitro transcribed (IVT) mRNA comprising a modified ribonucleotide can also be used for tailing IVT mRNA that is solely composed of unmodified ribonucleotides.

In parallel with the experiments described in Example 4, a tailing reaction was performed with IVT mRNA that was prepared solely with unmodified, naturally occurring ribonucleotides (ATP, GTP, CTP, and UTP). The tailing conditions were otherwise as described in Example 4. The concentrations of the reducing agent in buffer 8 were varied as described there. The resulting tailed unmodified IVT mRNA was analyzed by capillary gel electrophoresis. The results are summarized in Table 4.

TABLE 4

| DTT concentration | Untailed mRNA detected |
|---|---|
| 0 mM | No |
| 5 mM | No |
| 10 mM | No |
| 15 mM | No |
| 25 mM | No |
| 50 mM | No |

Under the test conditions of Example 4, no additional peak was discernible in any of the sample comprising unmodified IVT mRNA. Therefore, this example demonstrates that the reaction buffer of the invention can also be used for tailing IVT mRNA that is solely composed of unmodified ribonucleotides.

### Example 7. Large-Scale Tailing of Modified IVT mRNA

This example demonstrates that an optimized reaction buffer of the invention can be used for tailing of IVT mRNA comprising a modified ribonucleotide at a large scale (>1 g of IVT mRNA per batch).

The optimized reaction buffer identified in Example 4 (buffer 8 with 10 mM DTT) was used to tail four IVT mRNAs (mRNAs 1-4) with different nucleic acid sequences and lengths before tailing (1941, 1941, 1989 and 1995 nucleotides, respectively). Each batch comprised about 16 g of untailed IVT mRNA comprising a modified ribonucleotide (N1-methylpseudouridine in place of uridine). The IVT mRNA was prepared as described in Example 1. The tailing conditions were selected to add polyA tails to the IVT mRNAs with a length of 200 nucleotides (the desired tail length).

Capillary gel electrophoresis was used to determine the average length of the added polyA tails and presence of untailed mRNA. The results are summarized in Table 5.

TABLE 5

| mRNA | Tail length (nt) | Untailed mRNA detected |
|---|---|---|
| 1 | 208 | No |
| 2 | 203 | No |
| 3 | 205 | No |
| 4 | 254 | No |

As can be seen from Table 5, the average length of the polyA tails was close to the desired tail length of 200 nucleotides. No untailed species were detected.

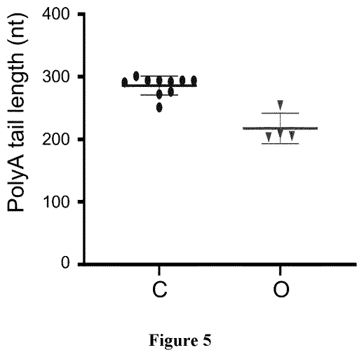

Using capillary gel electrophoresis, ten 10 g batches of corresponding tailing reactions performed with the unoptimized control buffer described in Table 2 were compared with the four 16 g batches prepared in this example. The average length of the added polyA tails was plotted on a graph as shown in FIG. 5. The average tail length obtained in tailing reactions using the unoptimized control buffer was closer to 300 nucleotides. As can be seen from FIG. 5, the average tail length achieved with the unoptimized control buffer (labelled as buffer ‘C’) was 45-50% longer than the desired tail length for most of the tested batches (9 out of 10). In contrast, using an optimized reaction buffer of the invention (labelled as buffer ‘O’), the average tail length was only about 5% longer than the desired tail length for three of the four tested batches. Even for the worst-performing batch, the average tail length was only about 25% longer than the desired tail length, when the optimized reaction buffer was used.

This example demonstrates that an optimized reaction buffer of the invention can be used to tail batches of IVT mRNA comprising a modified ribonucleotide at a large scale (>1 g). The resulting average tail length was close to the desired length (typically within 5% of the desired value). This example further confirms that the nucleic acid sequence of the IVT mRNA does not affect tailing efficiency when an optimized reaction buffer of the invention is used.

What is claimed is:

1. A method for tailing in vitro transcribed (IVT) messenger RNA (mRNA) comprising a modified ribonucleotide, said method comprising adding the IVT mRNA to a tailing polymerase in a reaction buffer comprising 30 mM or less of an alkali metal salt and 5 mM or more of a reducing agent.

2. The method of claim 1, wherein the IVT mRNA comprises a 5′ cap.

3. The method of claim 2, where the 5′ cap is added in a separate reaction, comprising a reaction buffer that is different to the reaction buffer of claim 1.

4. The method of any one of claims 1-3, wherein the alkali metal salt in the reaction buffer has a concentration of about 1 mM to about 30 mM.

5. The method of any one of claims 1-4, wherein the alkali metal salt in the reaction buffer has a concentration of about 5 mM or less.

6. The method of any one of the preceding claims, wherein the reducing agent in the reaction buffer has a concentration of 5 mM to 50 mM.

7. The method of claim 6, wherein the reducing agent in the reaction buffer has a concentration of 5 mM to 20 mM.

8. The method of claim 7, wherein the reducing agent in the reaction buffer has a concentration of about 10 mM.

9. The method of any one of the preceding claims, wherein the reducing agent is selected from dithiothreitol (DTT), 2-mercaptoethanol (2-ME), and tris(2-carboxyethyl)phosphine (TCEP).

10. The method of claim 9, wherein the reducing agent is dithiothreitol (DTT).

11. The method of any one of the preceding claims, wherein the alkali metal salt is NaCl or KCl.

12. The method of claim 11, wherein the alkali metal salt is NaCl.

13. The method of any one of the preceding claims, wherein the modified ribonucleotide is selected from pseudouridine, N1-methylpseudouridine, 5-methylcytidine, and 5-methoxyuridine.

14. The method of any one of the preceding claims, wherein the modified ribonucleotide is a modified uridine.

15. The method of claim 14, wherein the modified uridine is N1-methylpseudouridine.

16. The method of any one of the preceding claims, wherein at least 93% of the IVT mRNA is tailed.

17. The method of claim 16, wherein at least 94% of the IVT mRNA is tailed.

18. The method of claim 17, wherein at least 95% of the IVT mRNA is tailed.

19. The method of claim 18, wherein at least 96% of the IVT mRNA is tailed.

20. The method of claim 19, wherein at least 97% of the IVT mRNA is tailed.

21. The method of claim 20, wherein at least 98% of the IVT mRNA is tailed.

22. The method of any one of the preceding claims, wherein the mRNA tail comprises about 100 to about 800 ribonucleotides.

23. The method of claim 22, wherein the mRNA tail comprises about 100 to about 500 ribonucleotides.

24. The method of claim 23, wherein the mRNA tail comprises about 100 to about 250 ribonucleotides.

25. The method of claim 24, wherein the mRNA tail comprises about 100 or about 200 ribonucleotides.

26. The method of any one of the preceding claims, wherein the reaction buffer maintains a pH of about pH 7 to about pH 8.

27. The method of claim 26, wherein the reaction buffer maintains a pH of about pH 7.5.

28. The method of claim 26 or 27, wherein the reaction buffer maintains the pH with a buffering reagent selected from Tris, HEPES, MOPS, acetate, citrate, and phosphate.

29. The method of claim 28, wherein the buffering reagent is present at a concentration of about 5 mM to about 100 mM.

30. The method of claim 29, wherein the buffering reagent is present at a concentration of about 10 mM to about 50 mM.

31. The method of claim 30, wherein the buffering reagent is present at a concentration of about 50 mM.

32. The method of any one of the preceding claims, wherein the reaction buffer maintains the activity of the tailing polymerase by providing a divalent cation.

33. The method of claim 32, wherein the divalent cation is selected from Mg²⁺ and Mn²⁺.

34. The method of claim 32 or 33, wherein the divalent cation is present at a concentration of about 5 mM to about 20 mM.

35. The method of claim 34, wherein the divalent cation is at a concentration of about 5 mM to about 10 mM.

36. The method of claim 35, wherein the divalent cation is at a concentration of about 10 mM.

37. The method of any one of the preceding claims, wherein the IVT mRNA does not contain a modified ribonucleotide at the 3′ end.

38. The method of any one of the preceding claims, wherein the IVT mRNA does not contain a modified uridine at the 3′ end.

39. The method of any one of the preceding claims, wherein the IVT mRNA does not contain a N1-methylpseudouridine at the 3′ end.

40. The method of any one of the preceding claims, wherein the tailing polymerase is a polyA polymerase.

41. The method of claim 40, wherein the polyA polymerase is a bacterial polyA polymerase or a yeast polyA polymerase.

42. The method of claim 41, wherein the polyA polymerase is an E. coli polyA polymerase.

43. The method of any one of claims 40-42, wherein the reaction buffer includes a suitable concentration of ATP.

44. The method of claim 43, where ATP is present at a concentration of about 0.1 mM to about 10 mM.

45. A reaction buffer for use in a method for tailing in vitro transcribed (IVT) messenger RNA (mRNA) comprising 30 mM or less of an alkali metal salt, 5 mM or more of a reducing agent.

46. The method of claim 45, wherein the alkali metal salt in the reaction buffer has a concentration of about 1 mM to about 30 mM.

47. The reaction buffer of claim 45 or 46, wherein the alkali metal salt in the reaction buffer has a concentration of about 5 mM or less.

48. The reaction buffer of any one of claims 45-47, wherein the reducing agent in the reaction buffer has a concentration of 5 mM to 50 mM.

49. The reaction buffer of claim 48, wherein the reducing agent in the reaction buffer has a concentration of 5 mM to 20 mM.

50. The reaction buffer of claim 49, wherein the reducing agent in the reaction buffer has a concentration of about 10 mM.

51. The reaction buffer of any one of claims 45-50, wherein the reducing agent is selected from dithiothreitol (DTT), 2-mercaptoethanol (2-ME), and tris(2-carboxyethyl)phosphine (TCEP).

52. The reaction buffer of claim 51, wherein the reducing agent is dithiothreitol (DTT).

53. The reaction buffer of any one of claims 45-52, wherein the alkali metal salt is NaCl or KCl.

54. The reaction buffer of claim 53, wherein the alkali metal salt is NaCl.

55. The reaction buffer of any one of claims 45-54, wherein the reaction buffer has a pH of about pH 7 to about pH 8.

56. The reaction buffer of claim 55, wherein the reaction buffer has a pH of about pH 7.5.

57. The reaction buffer of claim 55 or 56, wherein the reaction buffer comprises Tris, HEPES, MOPS, acetate, citrate, or phosphate as a buffering reagent.

58. The reaction buffer of claim 57, wherein the buffering reagent is present at a concentration of about 5 mM to 100 mM.

59. The reaction buffer of claim 58, wherein the buffering reagent is present at a concentration of about 10 mM to about 50 mM.

60. The reaction buffer of claim 59, wherein the buffering reagent is present at a concentration of about 50 mM.

61. The reaction buffer of any one of claims 45-60, wherein the reaction buffer comprises a divalent cation.

62. The reaction buffer of claim 61, wherein the divalent cation is selected from Mg²⁺ and Mn²⁺.

63. The reaction buffer of claim 62, wherein the reaction buffer comprises MgCl₂ or MnCl₂.

64. The reaction buffer of any one of claims 45-63, wherein the divalent cation is present at a concentration of about 5 mM to about 20 mM.

65. The reaction buffer of claim 64, wherein the divalent cation is at a concentration of about 10 mM.

66. A composition comprising in vitro transcribed (IVT) messenger RNA (mRNA) in the reaction buffer of any one of claims 45-65.

67. A reaction buffer for use in a method for tailing in vitro transcribed (IVT) messenger RNA (mRNA) comprising 300 mM or less of an alkali metal salt, 50 mM or more of a reducing agent, wherein the buffer is diluted 10-fold prior to use.

68. The reaction buffer of claim 67, wherein the alkali metal salt in the reaction buffer has a concentration of about 10 mM to about 300 mM.

69. The reaction buffer of claim 67 or 68, wherein the alkali metal salt in the reaction buffer has a concentration of about 50 mM or less.

70. The reaction buffer of claim 69, wherein the reducing agent in the reaction buffer has a concentration of 50 mM to 500 mM.

71. The reaction buffer of claim 70, wherein the reducing agent in the reaction buffer has a concentration of 50 mM to 200 mM.

72. The reaction buffer of claim 71, wherein the reducing agent in the reaction buffer has a concentration of about 100 mM.

73. The reaction buffer of any one of claims 67-72, wherein the reducing agent is selected from dithiothreitol (DTT), 2-mercaptoethanol (2-ME), and tris(2-carboxyethyl)phosphine (TCEP).

74. The reaction buffer of claim 73, wherein the reducing agent is dithiothreitol (DTT).

75. The reaction buffer of any one of claims 67-74, wherein the alkali metal salt is NaCl or KCl.

76. The reaction buffer of claim 75, wherein the alkali metal salt is NaCl.

77. The reaction buffer of any of claims 67-76, wherein the reaction buffer has a pH of about pH 7 to about pH 8.

78. The reaction buffer of claim 77, wherein the reaction buffer has a pH of about pH 7.5.

79. The reaction buffer of claim 77 or 78, wherein the reaction buffer comprises Tris, HEPES, MOPS, acetate, citrate, or phosphate as a buffering reagent.

80. The reaction buffer of claim 79, wherein the buffering reagent is present at a concentration of about 50 mM to 1000 mM.

81. The reaction buffer of claim 80, wherein the buffering reagent is present at a concentration of about 100 mM to about 500 mM.

82. The reaction buffer of claim 81, wherein the buffering reagent is present at a concentration of about 500 mM.

83. The reaction buffer of any one of claims 67-82, wherein the reaction buffer comprises a divalent cation.

84. The reaction buffer of claim 83, wherein the divalent cation is selected from Mg²⁺ and Mn²⁺.

85. The reaction buffer of claim 84, wherein the divalent cation comprises MgCl₂ or MnCl₂.

86. The reaction buffer of any one of claims 83-85, wherein the divalent cation is present at a concentration of about 50 mM to about 200 mM.

87. The reaction buffer of claim 86, wherein the divalent cation is at a concentration of about 100 mM.

88. A method for generating an in vitro transcribed (IVT) messenger RNA (mRNA) comprising a modified ribonucleotide, the method comprising:
(i) preparing a DNA template wherein the terminal 3′ residue of the DNA template does not encode a modified ribonucleotide of the IVT mRNA;
and
(ii) transcribing the DNA template with an RNA polymerase in an in vitro transcription (IVT) reaction comprising the modified ribonucleotide.

89. The method of claim 88, wherein the DNA template is a circular vector comprising a restriction site.

90. The method of claim 89, wherein step (i) comprises cutting the circular vector at the restriction site to yield the terminal 3′ residue of the DNA template that does not encode a modified ribonucleotide of the IVT mRNA.

91. The method of claim 89 or 90, wherein the restriction site is cut by BspQI.

92. The method of any one of claims 88-91, further comprising a step of tailing the IVT mRNA.

93. The method of claim 92, wherein the step of tailing comprises adding the IVT mRNA to a tailing polymerase in a reaction buffer comprising 30 mM or less of an alkali metal salt and 5 mM or more of a reducing agent.

94. The method of claim 93, wherein the alkali metal salt in the reaction buffer has a concentration of 5 mM or less.

95. A method for tailing in vitro transcribed (IVT) messenger RNA (mRNA) comprising a modified ribonucleotide, comprising:
(i) providing untailed IVT mRNA which does not comprise the modified ribonucleotide at the 3′ end; and
(ii) adding a reaction buffer and a tailing polymerase.

96. The method of claim 95, wherein the reaction buffer comprises 30 mM or less of an alkali metal salt and 5 mM or more of a reducing agent.

97. The method of claim 96, wherein the alkali metal salt in the reaction buffer has a concentration of about 1 mM to about 30 mM.

98. The method of claim 96 or 97, wherein the alkali metal salt in the reaction buffer has a concentration of 5 mM or less.

99. The method of any one of claims 95-98, wherein the reducing agent in the reaction buffer has a concentration of 5 mM to 20 mM.

100. The method of any one of claims 95-99, wherein the reaction buffer comprises a divalent cation.

101. The method of claim 100, wherein the divalent cation is present at a concentration of about 5 mM to about 20 mM.
